(19) United States
(12) Patent Application Publication
(10) Pub. No.: US 2023/0272389 A1
(43) Pub. Date: Aug. 31, 2023

(54) CIRCULAR miRNA SPONGES

(71) Applicant: National University of Singapore, Singapore (SG)

(72) Inventors: Annadoray Lavenniah, Singapore (SG); Roger Foo, Singapore (SG); Matthew Ackers-Johnson, Singapore (SG)

(73) Assignee: National University of Singapore, Singapore (SG)

(21) Appl. No.: 18/014,315

(22) PCT Filed: Jul. 2, 2021
(86) PCT No.: PCT/SG2021/050385

§ 371 Date: Jan. 3, 2023

(30) Foreign Application Priority Data
Jul. 3, 2020 (SG) 10202006446X

(51) Int. Cl. C12N 15/113; C12N 15/11; A61P 9/04

(52) U.S. Cl. CPC C12N 15/113; C12N 15/111; A61P 9/04; C12N 2310/533; C12N 2310/113; C12N 2310/532; C12N 2310/51

(57) ABSTRACT

The present invention relates to miRNA interference technology. More specifically the invention relates to circular miRNA sponges that carry a plurality of binding sites directed to at least two types of miRNA and separated by random, non-identical spacers, allowing for the inhibition of functional classes of m1RNAs. Preferably, the binding sites are bulged binding sites wherein each bulge is created by a one base deletion and two base mismatch at positions 9-11 nt from the 3′ end of each binding site. Preferably, each spacer is 6 to 24 nucleotides in length. Preferably, the binding sites are against miR-132 and miR-212, miR-17-5p and miR-18a-5p, or miR-20b-5p and miR-106a-5p. Construction vectors and uses of said miRNA sponges for the treatment of diseases, such as cardiomyopathy and cancer, are also disclosed.

### FIELD OF THE INVENTION

The present invention relates to miRNA interference technology. More specifically the invention relates to engineered circular miRNA sponges that carry a plurality of binding sites directed to at least two types of miRNA and separated by non-identical spacers, allowing for the inhibition of functional classes of miRNAs; to construction vectors; and uses of said miRNA sponges for the treatment of diseases.

### BACKGROUND OF THE INVENTION

As fine-tuners of gene expression, miRNAs play essential roles in normal development and homeostasis, dysregulation of which has been implicated in the pathogenesis of various diseases. Hence, targeting candidate miRNAs presents an exploitable therapeutic avenue. The first anti-miRNA therapeutic drug, Miravirsen, has shown efficacy in phase II clinical trials against Hepatitis C Virus infection with minimal side effects [Janssen, H. L. A., et al. N. Engl. J. Med. 368: 1685-1694 (2013)]. While the continuous development and optimisation of existing miRNA interference technology has conferred therapeutic benefits in clinical trials, many challenges remain. These include short half-lives, off-target effects and potential accumulation of non-metabolisable molecules such as LNA nucleotides.

Circular RNAs (circRNAs) belong to an emerging class of noncoding RNA that exist in circular instead of canonical linear form. The cellular splicing mechanism plays a central role in the biogenesis of circRNAs from pre-mRNAs. circRNA are generated through a back-splicing reaction where the 5′ splice donor site of a downstream exon is fused to the 3′ splice acceptor site of an upstream exon. Recently, there has been emerging evidence of endogenous circRNAs functioning as miRNA sponges. The first two circRNAs to be elucidated as miRNA sponges were ciRS-7 and circSRY [Hansen, T. B., et al., Nature 495: 384-388 (2013)]. More than 70 seed sites for miR-7 were identified in ciRS-7, and 16 miR-138 sites were identified in circSRY. Of note, following miR-7 binding, ciRS-7 levels remained unaffected, whereas co-tested linear miR-7 sponge constructs saw an approximate 2-fold decrease in abundance, presumably the result of exonucleolytic degradation. Thus, the circularity of the circRNA may confer resistance to degradation upon miRNA binding [Hansen, T. B., et al., Nature 495: 384-388 (2013)]. circRNAs are also protected against RNase-mediated exonucleolytic decay due to non-requirement of a 5′ cap and a 3′ polyA tail [Ebbesen, K. K., et al., Biochim. Biophys. Acta 1859: 163-168 (2016)]. This is consistent with the typical observation of deadenylation and decapping when miRNA bind to their canonical linear mRNA targets [Ebbesen, K. K., et al., Biochim. Biophys. Acta 1859: 163-168 (2016)].

An example of an application for circRNAs is in the prophylaxis or treatment of heart failure, which is the final common pathology for a myriad of cardiovascular diseases such as hypertension, metabolic syndrome, valve disease and others [Creemers, E. E., et al., Nat. Rev. Genet. 12: 357-362 (2011)]. It is a major cause of mortality and morbidity worldwide and poses a significant healthcare burden, with the pressing need for novel therapeutic approaches.

Pathological hypertrophy associates with cardiac dysfunction and fibrotic remodelling, leading to wall stiffness, which compromises systolic and diastolic function, and ultimately progresses to heart failure. Cardiac hypertrophy is one of the strongest prognostic factors in patients with heart disease, and reduction in pathological hypertrophy or adverse myocardial remodelling represents a therapeutic goal for cardiovascular pharmacotherapy. It was recently shown that miR-212/132 family expression is upregulated in cardiomyocytes following hypertrophic stimuli, and miR-212/132 necessarily drive pathological hypertrophy [Ucar, A., et al., Nat. Commun. 3: 1078 (2012)]. Pharmacological targeting of miR-132 by antagomiRs reduces cardiac hypertrophy progression, and abrogates heart failure in rodent models [Ucar, A., et al., Nat. Commun. 3: 1078 (2012)], and is currently entering Phase I clinical trials [Foinquinos, A., et al., Nat. Commun. 11: 1-10 (2020)].

There is a need to provide improved miRNA interference technology that could be used in pharmaceutical drugs and therapy.

### SUMMARY OF THE INVENTION

In this application, the inventors constructed a circular miRNA sponge, termed “circmiR”, which was engineered as a custom sponge to sequester target miRNAs of interest. As proof of concept, the mouse pressure-overload induced cardiac hypertrophy disease model was chosen to test the circmiR of the invention. Hence, the circmiR was designed to target the miR-212/132 family to test its effectiveness as a miRNA inhibitor compared with the current gold standard antagomir technology as a new development for pharmacotherapy.

The present invention optimises crucial parameters in engineered circmiRs such as the number and type of binding sites to be incorporated, as well as between-site spacer sizes. Cardiomyocyte-specific in vivo delivery of optimised circmiRs, targeting the miR-212/132 family, attenuated left ventricular hypertrophy. Furthermore, circmiRs exhibited improved efficacy, compared to equimolar pharmacological antagomirs in vitro, and enhanced stability compared to linear counterparts. To the best of our knowledge, the present invention introduces the first instance of therapeutic application of a targeted miRNA interference technology, circmiRs, in vivo. The optimum parameters were determined for other miRNA targets to determine a set of optimized parameters applicable when constructing circmiRs to a target.

The present invention provides an isolated circular RNA polynucleotide microRNA sponge, comprising:
a) a plurality of human or non-human animal microRNA bulged binding sites, wherein each binding site comprises a region which is 100% complementary to a microRNA seed region;
b) a plurality of polynucleotide spacers, wherein each spacer is of eight to twenty nucleotides and positioned between two binding sites;
wherein said plurality of spacers comprises at least two spacers having a random, non-identical, sequence.

In some embodiments, the isolated circular RNA polynucleotide microRNA sponge comprises five to ten of each of two different microRNA bulged binding sites. For example, the microRNA sponge may comprise an even number of binding sites, such as 6, 10, 12, 14, 16, or more binding sites.

In some embodiments, the circular RNA polynucleotide contains a total of 12 microRNA binding sites.

In some embodiments, the isolated circular RNA polynucleotide contains spacers of 6 to 24 nucleotides, preferably 12 nucleotides, in length between microRNA binding sites.

In some embodiments, the different microRNA binding sites are alternated in the circular RNA polynucleotide.

In some embodiments, the bulge in each respective binding site is created by a one base deletion and two base mismatches at positions 9-11 nt from the 3′ end of each binding site.

In some embodiments, the isolated circular RNA polynucleotide comprises:
i) human or non-human animal miR-132 microRNA bulged binding sites and human or non-human animal miR-212 microRNA bulged binding sites; or
ii) human or non-human animal miR-17-5p microRNA bulged binding sites and human or non-human animal miR-18a-5p microRNA bulged binding sites; or
iii) human or non-human animal miR-20b-5p microRNA bulged binding sites and human or non-human animal miR-106a-5p microRNA bulged binding sites,
wherein each binding site comprises a region which is 100% complementary to the microRNA seed region and is separated by a polynucleotide spacer of eight to twenty nucleotides which comprises a random, non-identical, sequence to reduce repetition of sequences within the sponge and wherein binding sites directed to different microRNA are alternated to reduce repetition of sequences within the sponge.

In some embodiments, the isolated circular RNA polynucleotide comprises twelve bulged binding sites selected from the group comprising, six miR-132 bulged binding sites alternating with six miR-212 bulged binding sites; six miR-17-5p bulged binding sites alternating with six miR-18a-5p bulged binding sites; and six miR-20b-5p bulged binding sites alternating with six miR-106a-5p bulged binding sites; and spacers of twelve nucleotides between each binding site.

In some embodiments, the isolated circular RNA polynucleotide comprises bulged binding sites, wherein:
i) respective binding sites are complementary to the miR-132 microRNA nucleic acid sequence set forth in 3′-GCUGGUACCGACAUCUGACAAU-5′ SEQ ID NO: 1 and complementary to the miR-212 microRNA nucleic acid sequence set forth in 3′-ACCGGCACUGACCUCUGACAAU-5′ SEQ ID NO: 2; or
ii) respective binding sites are complementary to the miR-17-5p microRNA nucleic acid sequence set forth in 3′-CAAAGUGCUUACAGUGCAGGUAG-5′ SEQ ID NO: 5 and complementary to the miR-18a-5p microRNA nucleic acid sequence set forth in 3′-UAAGGUGCAUCUAGUGCAGAUAG-5′ SEQ ID NO: 6; or
iii) respective binding sites are complementary to the miR-20b-5p microRNA nucleic acid sequence set forth in 3′-CAAAGUGCUCAUAGUGCAGGUAG-5′ SEQ ID NO: 9 and complementary to the miR-106a-5p microRNA nucleic acid sequence set forth in 3′-AAAAGUGCUUACAGUGCAGGUAG-5′ SEQ ID NO: 10.

In some embodiments, the isolated circular RNA polynucleotide comprises bulged binding sites, wherein:
i) the bulged binding site for miR-132 comprises the nucleic acid sequence set forth in 5′-CGACCAUGGCUCAGACUGUUA-3′ SEQ ID NO: 3 and the bulged binding site for miR-212 comprises the nucleic acid sequence set forth in 5′-UGGCCGUGACUCCGACUGUUA-3′ SEQ ID NO: 4;
ii) the bulged binding site for miR-17-5p comprises the nucleic acid sequence set forth in 3′-CUACCUGCACUGAUGCACUUUG-5′ SEQ ID NO: 7 and the bulged binding site for miR-18a-5p comprises the nucleic acid sequence set forth in 3′-CUAUCUGCACUACUGCACCUUA-5′ SEQ ID NO: 8; or
iii) the bulged binding site for miR-20b-5p comprises the nucleic acid sequence set forth in 3′-CUACCUGCACUAACGCACUUUG-5′ SEQ ID NO: 11 and the bulged binding site for miR-106a-5p comprises the nucleic acid sequence set forth in 3′-CUACCUGCACUGAUGCACUUUU-5′ SEQ ID NO: 12.

In some embodiments, the isolated circular RNA polynucleotide comprises the nucleic acid sequence selected from the nucleic acid sequences set forth in the group comprising:
i) SEQ ID NO: 13, comprising 6 copies of SEQ ID NO: 3 alternating with 6 copies of SEQ ID NO: 4, and 2 copies of spacers SEQ ID NO: 27, 28, 29, 30 and 31 and 1 copy of spacer SEQ ID NO: 32;
ii) SEQ ID NO: 14, comprising 6 copies of SEQ ID NO: 7 alternating with 6 copies of SEQ ID NO: 8, and 2 copies of spacers SEQ ID NO: 27, 28, 29, 30 and 31 and 1 copy of spacer SEQ ID NO: 32; and
iii) SEQ ID NO: 15, comprising 6 copies of SEQ ID NO: 11 alternating with 6 copies of SEQ ID NO: 12, and 2 copies of spacers SEQ ID NO: 27, 28, 29, 30 and 31 and 1 copy of spacer SEQ ID NO: 32.

In some embodiments, the isolated circular RNA polynucleotide can be used as a medicament. For example, the circular RNA polynucleotide may be used in the treatment of cardiomyopathy, if the circular RNA polynucleotide comprises miR-132 bulged binding sites and miR-212 bulged binding sites.

For example, the circular RNA polynucleotide may be used in the treatment of cancer, if the circular RNA polynucleotide comprises miR-17-5p bulged binding sites and miR-18a-5p bulged binding sites, or comprises miR-20b-5p bulged binding sites and miR-106a-5p bulged binding sites.

In a second aspect, the invention provides a pharmaceutical composition comprising a circular RNA polynucleotide; and at least one of a pharmaceutically acceptable diluent, carrier and adjuvant.

In a third aspect, the invention provides an isolated DNA expression construct comprising a nucleic acid sequence encoding the circular RNA polynucleotide, operably linked to a promoter, inverted complementary introns flanking the RNA polynucleotide microRNA sponge sequence, a splice acceptor site (SA) and a splice donor site (SD).

In some embodiments. the expression construct comprises a nucleic acid sequence encoding the circular RNA polynucleotide of any aspect of the invention. In some embodiments, the expression construct comprises a nucleic acid sequence encoding the circular RNA polynucleotide set forth in SEQ ID NO: 13, SEQ ID NO: 14, or SEQ ID NO: 15.

In a fourth aspect, the invention provides an expression vector comprising the DNA expression construct.

In some embodiments, the expression vector comprises a constitutive promoter such as CMV, CAG or EF-1 alpha or an inducible promoter such as TRE, or a cardiac- or cardiomyocyte-specific promoter.

In some embodiments, the promoter is selected from the group comprising a cardiac troponin T promoter (cTnT), an α-myosin heavy chain (α-MHC) promoter and a myosin light chain (MLC2v) promoter.

In some embodiments, the expression vector is a virus expression vector, preferably selected from the group comprising Lentivirus, Adenovirus and Adeno-associated virus (AAV).

In a fifth aspect, the invention provides an isolated circular RNA polynucleotide, pharmaceutical composition, expression construct or expression vector of any aspect of the invention for the treatment, amelioration or prevention of a disease or medical disorder associated with the presence or over-expression of a plurality of microRNA.

In some embodiments, the plurality of microRNA comprises miR-132 and miR-212, miR-17-5p and miR-18a-5p, or miR-20b-5p and miR-106a-5p.

In some embodiments, the disease or medical disorder is:
a) cardiomyopathy, when the circular RNA polynucleotide comprises miR-132 bulged binding sites and miR-212 bulged binding sites; or
b) cancer, when the circular RNA polynucleotide comprises miR-17-5p bulged binding sites and miR-18a-5p bulged binding sites, or comprises miR-20b-5p bulged binding sites and miR-106a-5p bulged binding sites.

In a sixth aspect, the invention provides a use of a circular RNA polynucleotide, pharmaceutical composition, expression construct, or expression vector of any aspect of the invention for the manufacture of a medicament for the treatment, amelioration or prevention of a disease or medical disorder associated with the presence or over-expression of a plurality of microRNA.

In some embodiments, the microRNA comprises miR-132 and miR-212, miR-17-5p and miR-18a-5p, or miR-20b-5p and miR-106a-5p.

In some embodiments, the disease or medical disorder is cardiomyopathy or cancer.

In a seventh aspect, the invention provides a method for the treatment, amelioration or prevention of a disease or medical disorder associated with the presence or over-expression of a plurality of microRNA, comprising the step of administering an efficacious amount of a circular RNA polynucleotide, pharmaceutical composition, expression construct, or expression vector of any aspect of the invention to a human or non-human animal in need of such treatment.

In some embodiments, the microRNA comprises miR-132 and miR-212, miR-17-5p and miR-18a-5p, or miR-20b-5p and miR-106a-5p.

In some embodiments, wherein the disease or medical disorder is cardiomyopathy or cancer.

In an eighth aspect, the invention provides a method of optimizing the structure of a circular RNA polynucleotide microRNA sponge comprising a plurality of bulged binding sites directed to human or non-human animal target miRNA, comprising the steps;
a) test the effect of a plurality of spacers of 6 to 24 nucleotides in length between binding sites, in a circular RNA polynucleotide microRNA sponge comprising a plurality of bulged binding sites directed to one or more human or non-human animal target miRNA, on the binding to their target miRNA, and select the optimum spacer length;
b) test the effect of the number of binding sites from at least 6 in total, in a circular RNA polynucleotide microRNA sponge comprising a plurality of bulged binding sites directed to one or more human or non-human animal target miRNA, on the binding to their target miRNA;
c) engineer a circular RNA polynucleotide microRNA sponge comprising the optimum spacer length and number of binding sites from a) and b),
wherein said plurality of spacers comprises at least two spacers having a random, non-identical, sequence.

In some embodiments, the sponge comprises alternating binding sites.

In a ninth aspect, the invention provides an isolated circular RNA polynucleotide microRNA sponge produced according to the abovementioned method.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### BRIEF DESCRIPTION OF THE FIGURES

FIGS. 1A-F show the engineering of a circular miRNA sponge. (A) Design of a perfect complementary or imperfect bulged miRNA binding site. The bulge is created by one base deletion and two base mismatches at positions 9-11 nt. Seed regions are highlighted in yellow. (B) Schematic illustration of miRNA sponge construct carrying 12 binding sites separated by 6 nt spacers. (C) Schematic illustration of circmiR expression construct, indicating positions of the convergent (grey arrows) and circmiR-specific divergent (black arrows) PCR primer binding sites. (D) Sanger sequencing of PCR product following amplification with divergent circmiR primers confirming back-splicing of the miRNA sponge construct. (E) Schematic of varying length of intronic sequences, short and long, flanking the miRNA sponge sequence. (F) Expression abundance of circRNA derived from constructs with short or long flanking intronic sequences in transfected HEK293T cells using qPCR. (n=3); **P<0.01, ***P<0.001. Student's t-test.

FIGS. 2A-E show engineered circmiRs are efficient sponges of miR-132 and -212. Luciferase rescue reporter assays using dual reporter constructs with miR-132 and -212 binding sites inserted into the 3′UTR of the Renilla luciferase gene. HEK293T cells were co-transfected with dual reporter plasmid psiCheck2, miR-132 and -212 mimics and respective circRNA expression constructs for 48 hours, to determine (A) the effect of circmiR versus circScram, (B) the effect of circmiR with different spacer lengths: 6, 12, 24, 36, 72 nucleotides, (C) the effect of circmiR with different numbers of miRNA binding sites: 2, 6, 8, 12, 16, and (D) the effect of bulged versus perfect complementary miRNA binding sites. RNA hybrid prediction of structures are shown for each binding site type upon miRNA binding. (n=3); *P<0.05, **P<0.01, ***P<0.001, ****P<0.0001 relative to control with mimics. One-way ANOVA with Benjamini-Hochberg adjustment. (E) Expression abundance 48 hours post-transfection of circmiRs carrying either 12 bulged or perfect miRNA binding sites in the presence or absence of mimics in HEK293T cells using qPCR. (n=3); **P<0.01 relative to control without mimics. Student's t-test.

FIG. 3 shows the efficacy of engineered circmiRs in H9C2 cardiomyocytes. Luciferase rescue reporter assays using dual reporter constructs with miR-132 and -212 binding sites inserted into the 3′UTR of Renilla. H9C2 cardiomyocytes were co-transfected with the dual reporter plasmid psiCheck2, miR-132 and -212 mimics and respective circRNA expression constructs, to determine the effect of bulged circmiR or perfect circmiR versus circScram. (n=3). ****P<0.0001 relative to control with mimics. One-way ANOVA with Benjamini-Hochberg adjustment.

FIG. 4 shows that cardiac miR-132 and miR-212 levels were upregulated in left ventricular pressure-overloaded mice. Expression levels of miR-132 and miR-212 in mice ten to eleven weeks after TAC (n=7) or sham (n=6) surgery. FC: Fold change. P<0.05, **P<0.01 relative to sham. Student's t-test.

FIGS. 5A-H show that circmiR therapy attenuates pressure overload induced hypertrophy. (A) Experimental strategy to test circmiR therapeutic or circScram/linsp control constructs in vivo by AAV9 injection to 7-week-old mice. TAC surgery was performed one week later. Weekly echocardiography was conducted up to 4 weeks post TAC surgery before sacrifice. (B) Expression abundance of products from AAV construct circScram, linsp, circmiR in isolated cardiomyocytes using qPCR. (C-E) Echocardiographic analysis of (C) ejection fraction, (D) interventricular septal thickness, and (E) left ventricular posterior wall thickness in circScram, linsp, circmiR injected mice and sham-operated controls. (F) Heart weight to tibia length ratios and (G) cardiomyocyte diameter measured by immunofluorescence analysis of WGA and cTnl staining to visualise cell membrane and cardiomyocytes respectively in circScram, linsp, circmiR injected mice and sham-operated controls. (H) Expression levels of cardiac stress response genes Nppa, Nppb, Myh7/Myh6 ratio in isolated cardiomyocytes using qPCR. (I) miR-132 and miR-212 expression levels in isolated cardiomyocytes using qPCR. For all data, circScram (n=12), circmiR (n=11), linsp (n=11) and sham (n=9) except (F) and (G) where circScram (n=10), circmiR (n=11), linsp (n=10) and sham (n=7). *P<0.05, **P<0.01, ***P<0.001, ****P<0.0001 (sham versus circScram). #P<0.05, ##P<0.01, ###P<0.001 (circScram versus circmiR/linsp). One-way ANOVA with Benjamini-Hochberg adjustment.

FIG. 6 shows primer design to distinguish between linear and circular forms of miRNA sponge. Schematic illustration of AAV circmiR and linear sponge expression constructs, indicating positions of the circmiR-specific divergent (black arrows) and linear sponge-specific convergent (white arrows) PCR primer binding sites.

FIG. 7 shows that administration of circmiR or linear sponge leads to reduced miR-132/212 abundance in HEK293T cells. Expression levels of miR-132 and miR-212 in HEK293T cells 48 h after transfection FC: Fold change. (n=3); ****P<0.0001 relative to circScram. One-way ANOVA with Benjamini-Hochberg adjustment.

FIGS. 8A-B show that circRNA offers superior protection against endogenous nuclease degradation. RNA stability assay of sponge constructs in HEK293T cells following actinomycin D treatment. 18S and MYC act as endogenous control transcripts while mCherry is a plasmid driven control transcript. RNA levels at each time point were measured by qPCR, to enable comparison of RNA stability between (A) plasmid-driven circmiRs versus linear sponges, and (B) in vitro synthesised circmiRs versus linear sponges. **P<0.01, ****P<0.0001 (circmiR versus linear sponge). One-way ANOVA with Benjamini-Hochberg adjustment.

FIGS. 9A-C show 12 alternating bulged binding sites with 12 nt spacers, where at least 2 spacers are non-identical, generate efficient sponges of miR-17 and -18; and miR-20b and -106a. Luciferase rescue reporter assays using dual reporter constructs with either miR-17 and -18a or miR-20b and miR-106a binding sites inserted into the 3′-UTR of the Renilla luciferase gene. HEK293T cells were co-transfected with dual reporter plasmid psiCheck2 and respective circRNA expression constructs for 48 h, to determine (A) the effect of circmiR with different spacer lengths: 6, 12, 24, 36, 72 nt, (B) the effect of bulged versus perfect complementary miRNA binding sites and (C) the effect of circmiR with different numbers of miRNA binding sites: 2, 6, 8, 12, 16. The relative levels of Renilla luciferase normalised to Firefly luciferase are plotted. Error bars represent SEM (n=3). *P<0.05, **P<0.01, ***P<0.001, ****P<0.0001 relative to Luc 3′ UTR_miR. #P<0.05, ####P<0.0001 (perfect circmiR versus bulged circmiR). One-way ANOVA with Benjamini-Hochberg adjustment.

### DETAILED DESCRIPTION OF THE INVENTION

Bibliographic references mentioned in the present specification are for convenience listed at the end of the examples. The whole content of such bibliographic references is herein incorporated by reference.

### Definitions

Certain terms employed in the specification, examples and appended claims are collected here for convenience.

As used in the specification and the appended claims, the singular forms “a,” “an” and “the” include plural referents unless the context clearly dictates otherwise.

As used herein, ranges can be expressed as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that each unit between two particular units are also disclosed. For example, if 10 and 15 are disclosed, then 11, 12, 13, and 14 are also disclosed.

As used herein, the term “miRNA” and “microRNA” are interchangeable. miRNA refers to a small single-stranded non-coding RNA molecule that functions in RNA silencing and post-transcriptional regulation of gene expression. In the examples, “miRNA” may be further abbreviated as “miR”.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. Nucleic acid molecules can be naturally occurring, recombinant, or synthetic. The term “nucleotide” may be further abbreviated as “nt”.

As used herein, the term “oligonucleotide”, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimers,” “primers,” “oligomers,” and “probes,” as these terms are commonly defined in the art.

As will be appreciated by those of skill in the art, in certain embodiments, the nucleic acid further comprises a plasmid sequence. The plasmid sequence can include, for example, one or more sequences of a promoter sequence, a selection marker sequence, or a locus-targeting sequence. Methods of introducing nucleic acid compositions into cells are well known in the art.

It would be understood that oligonucleotides used in the present invention may be structurally and/or chemically modified to, for example, prolong their activity in samples potentially containing nucleases, during performance of methods of the invention, or to improve shelf-life in a kit. Thus, the circular miRNA sponge or any oligonucleotide primers or probes used according to the invention may be chemically modified. In some embodiments, said structural and/or chemical modifications include the addition of tags, such as fluorescent tags, radioactive tags, biotin, a 5′ tail, the addition of phosphorothioate (PS) bonds, 2′-O-Methyl modifications and/or phosphoramidite C3 Spacers during synthesis.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

### EXAMPLES

A person skilled in the art will appreciate that the present invention may be practiced without undue experimentation according to the methods given herein. The methods, techniques and chemicals are as described in the references given or from protocols in standard biotechnology and molecular biology textbooks. Standard molecular biology techniques known in the art and not specifically described were generally followed as described in Sambrook and Russel, Molecular Cloning: A Laboratory Manual, Cold Springs Harbor Laboratory, New York (2001).

### Example 1

### Materials and Methods

### Cell Culture and Transfections

HEK293T and H9C2 cells were maintained in DMEM (GE Healthcare Life Science) supplemented with 10% FBS (Capricorn Scientific), 100 U/ml penicillin and 100 μg/ml streptomycin (Nacalai Tesque, Inc) in a humidified atmosphere at 37° C., 5% CO₂. Transient transfection of plasmids and/or miRNA mimics (Qiagen) into both cell lines was carried out with Lipofectamine 2000 reagent (Invitrogen) according to manufacturer's protocol.

circmiR Construct Design

The miRNA sponge sequence was constructed using Ultramer DNA oligos (Integrated DNA Technologies (IDT)) designed to contain either 2, 4, or 6 miRNA binding sites. Binding sites were designed as the reverse complement of the mature sequences of mmu-miR-132-3p or mmu-miR-212-3p (miRBase). Bulged sites carried one deletion and two base mismatches outside the seed regions as described [Gentner, B., Schira, G., Giustacchini, A., Amendola, M., Brown, B. D., Ponzoni, M., and Naldini, L. (2009) Nat. Methods 6, 63-66], while perfect sites had complete complementarity to the mature miRNA sequences (Table 1). Spacers of different lengths having random, non-identical, sequences (such as spacers having polynucleotide sequences set forth in SEQ ID NOs: 27, 28, 29, 30, 31 and 32) and a scrambled sequence (circScram) were created using a random oligo generator (mkwakDOTorg/oligorand/). The online RNAhybrid tool [Kruger, J., and Rehmsmeier, M. (2006) Nucleic Acids Res. 34, W451-W454; Rehmsmeier, M., Steffen, P., Hochsmann, M., and Giegerich, R. (2004) RNA 10, 1507-1517] was used to confirm binding of designed miRNA sponge sequences to target miR-212/132 sequences.

1.1 kb of the 5′ intronic sequence upstream and 1.1 kb of the 3′ intronic sequence downstream of exon 2 of the mouse Slc8a1 locus (encoding a known Slc8a1 circRNA) was PCR-amplified using primers shown in Table 1. Both fragments were cloned into an AAV9-cTnT-eGFP plasmid backbone (gift from Dr. Jiang Jianming, Cardiovascular Research Institute, National University of Singapore). The miRNA sponge oligos were cloned between these fragments to generate circmiRs carrying 2 or 6 binding sites. For circmiRs carrying 8, 12 or 16 binding sites, additional miRNA sponge oligos were sequentially cloned according to Blachinsky, E., et al., [BioTechniques 36: 933 (2004), incorporated herein by reference] between the inverted intronic repeats. The linear sponge sequence comprised of 12 bulged miRNA binding sites, constructed by sequential cloning [Blachinsky, E., et al., BioTechniques 36: 933 (2004)] of miRNA sponge oligos carrying 6 binding sites into the AAV9-cTnT-eGFP plasmid, without inverted intronic repeats. circScram was constructed by inserting a scrambled sequence, ordered as a gBlock gene fragment (IDT), between the inverted intronic repeats in the AAV9-cTnT-eGFP plasmid. The entire circmiR/linear/circScram sponge sequences were cloned into pCAG-mCherry plasmid backbones for in vitro applications.

TABLE 1
Primer sequences used in cloning.

| | | SEQ |
|---|---|---|
| Cloning | | ID |
| constructs | Forward/Reverse | NO: |
| 5′ Intron | 5′ -TAGAATCGCCACTCCTGCAT -3′ | 19 |
| | 5′- TTGGGTGGGAGACTTAATCG -3′ | 20 |
| 3′ Intron | 5′- GAGGTGGAGGGGAAGACTTT -3′ | 21 |
| | 5′- TAGAATCGCCACTCCTGCAT -3′ | 22 |
| miR-132 perfect | 5′- CGACCATGGCTGTAGACTGTTA -3′ | 23 |
| binding site | | |
| miR-132 bulged | 5′- CGACCATGGCTCAGACTGTTA -3′ | 24 |
| binding site | | |
| miR-212 perfect | 5′-TGGCCGTGACTGGAGACTGTTA-3′ | 25 |
| binding site | | |
| miR-212 bulged | 5′- TGGCCGTGACTCCGACTGTTA -3′ | 26 |
| binding site | | |

The presence of repeat sequences results in a high frequency of recombination events that interferes with techniques used to construct the sponge such as PCR, cloning and Sanger sequencing. While the bases in the non-seed interaction region of miRNAs could be altered to introduce randomization and prevent repetitiveness, studies have shown that the non-seed interaction region is important. Pairing outside the seed region allows for differential gene targeting. Despite harbouring the same seed sequence, miR-132 and miR-212 exert different roles in endothelial biology depending on their pairing with additional 3′ sequences present within target sites that allow for specific targeting [Kumarswamy, R., et al., European heart journal 35: 3224-3231 (2014)]. Thus, it is important to preserve the sequence of miRNA binding site as much as possible to bind each different miRNA. Instead, altering the binding sites for each different miRNA and randomizing the sequences of the spacers were carried out.

We introduced randomisation of bases in the spacers. A total of eleven 12 nt spacers were used in one trial (Table 2) and of these, five 12 nt spacer sequences were used twice at different positions and one 12 nt spacers sequence was used once.

TABLE 2
12 nt spacers with non-identical sequences

| Random 12 nt Spacer | SEQ ID NO. |
|---|---|
| 5′-TCAGCAGCTGTG-3′ | 27 |
| 5′-ACTCCTATCGTA-3′ | 28 |
| 5′-CTATAAGAAGCA-3′ | 29 |
| 5′-ACTCGCTATTAG-3′ | 30 |
| 5′-TCATGACTACCG-3′ | 31 |
| 5′-ATTCGGAGATCC-3′ | 32 |

Luciferase Reporter Assays

miRNA-132/212 binding sites were cloned downstream of the Renilla luciferase gene of the psiCHECK-2 vector (Promega). 5×10⁴ HEK293T cells were co-transfected with 50 ng of the psiCHECK-2-miR-212/132 or empty psiCHECK-2 vector, 50 ng of circmiR/circScram vectors and 10 μmol of equal molar mix of miR-212/132 mirVana™ miRNA mimics (Qiagen), using Lipofectamine™ 2000 reagent (Invitrogen) according to manufacturer's instructions. T7 synthesised circmiRs or miRvana™ miRNA inhibitors (Qiagen) were also co-transfected with μmol of equal molar mix of miR-212/132 mirVana™ miRNA mimics (Qiagen). H9C2 cells (a kind gift from Dr Zhou Yue, Cardiovascular Research Institute, National University of Singapore) were co-transfected with 50 ng of psiCHECK-2-miR-212/132 or empty psiCHECK-2 vector, 200 ng of circmiR/circScram vectors and 0.2 μmol of equal molar mix of miR-212/132 mirVana™ miRNA mimics (Qiagen). Luciferase activity was measured 48 h after transfection using the Dual-Glo™ Luciferase Assay System (Promega) according to manufacturer's protocol and read on a GloMax™ multi-plate reader (Promega). Results are expressed as Renilla luciferase normalised against Firefly luciferase.

RNA Isolation, cDNA Synthesis, and Real-Time qPCR

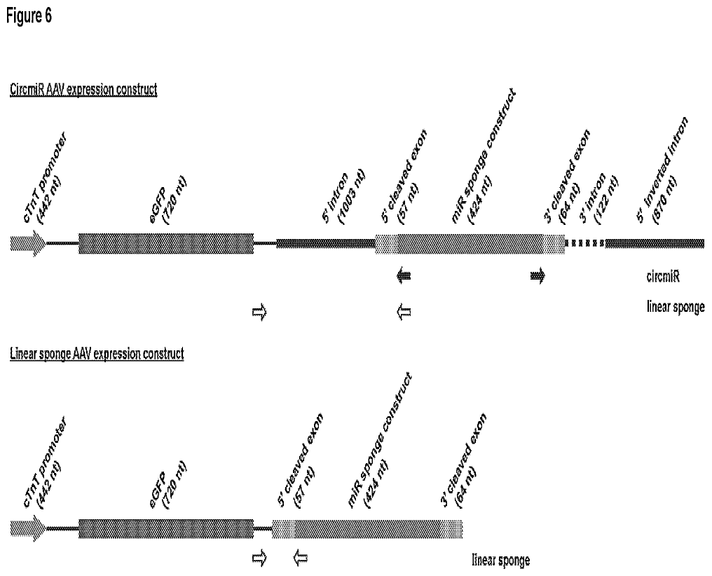

Total RNA was extracted from either isolated adult mouse cardiomyocytes or HEK293T cells using TRIzol™ Reagent (Thermo Fisher Scientific) according to standard procedures. Complementary DNA (cDNA) was synthesised with random primers using the gScript™ cDNA Synthesis Kit (Quantabio). Quantitative PCR (qPCR) was performed with Perfecta™ SYBR™ Green FastMix™ (Quantabio) on a LightCycler™ 480 (Roche) according to manufacturers' instructions using primers listed in Table 3. Primer design to distinguish between circmiR and linear sponge has been detailed in FIG. 6. All qPCR data were normalised to expression of the housekeeping genes GAPDH (for human genes) or 18S (for mouse genes). miRNA reverse transcription reactions were carried out using the miRCURY LNA™ Universal RT Kit (Qiagen) according to manufacturer's protocol. qPCR was performed with Perfecta™ SYBR™ Green FastMix™ (Quantabio) on a Rotor-Gene™ Q cycler (Qiagen) using miRCURY LNA™ miRNA PCR assay (Qiagen) primer sets for mmu-miR-132-3p and mmu-miR-212-3p. Results were normalised to expression of 18S. All qPCR reactions were carried out in duplicates.

TABLE 3
Primer sequences used in qPCR.

| | | | SEQ |
|---|---|---|---|
| | | | ID |
| Gene | Species | Forward/Reverse | NO: |
| GAPDH | Human | 5′- AGCCACATCGCTCAGACACC -3′ | 33 |
| | | 5′- GCCCAATACGACCAAATCC -3′ | 34 |
| 18S | Mouse | 5′- TTGACGGAAGGGCACCACCAG -3′ | 35 |
| | | 5′- GCACCACCACCCACGGAATCG -3′ | 36 |
| Nppa | Mouse | 5′- TCGGAGCCTACGAAGATCCA -3′ | 37 |
| | | 5′- GTGGCAATGTGACCAAGCTG -3′ | 38 |
| Nppb | Mouse | 5′- GCTGCTGGAGCTGATAAGAGAA -3′ | 39 |
| | | 5′- AGGTCTTCCTACAACAACTTCAGTG -3′ | 40 |
| Myh6 | Mouse | 5′- CTACAAGCGCCAGGCTGAG -3′ | 41 |
| | | 5′- TGGAGAGGTTATTCCTCGTCG -3′ | 42 |
| Myh7 | Mouse | 5′- AGCATTCTCCTGCTGTTTCCTT -3′ | 43 |
| | | 5′- TGAGCCTTGGATTCTCAAACG -3′ | 44 |

### TAC Model and Cardiomyocyte Isolation

All animal procedures were approved by the National University of Singapore Institutional Animal Care and Use Committee and were undertaken in strict accordance with Singapore National Advisory Committee for Laboratory Animal Research guidelines. Adult mice were housed in individually ventilated cages, with sex-matched littermates, under standard conditions. Food and water were available ad libitum. TAC or sham surgery was performed on 8-week-old male C57BL/6 mice as previously described [Rockman, H. A., et al., Proc. Natl. Acad. Sci. U.S.A 88: 8277-8281 (1991), incorporated herein by reference]. Left ventricular cardiomyocytes were isolated as previously described [Ackers-Johnson, M., et al., Circ. Res. 119: 909 (2016), incorporated herein by reference] from AAV-treated mice 4 weeks after surgery. Transthoracic echocardiography was performed according to manufacturer's guide for small animal echocardiography (Vevo™ 2100 Imaging System, Visualsonics). Doppler velocity measurements of right and left carotid arteries across the aortic constriction was performed at weeks 1 and 4 post-TAC mice to confirm the consistency of the surgical procedure (Vevo™ 2100 Imaging System, Visual Sonics).

### AAV9 Viral Production and Purification

circScram, circmiRs and linear sponge constructs were cloned into AAV9-cTnT-eGFP vectors as described above. The target AAV9 vectors were packaged by a triple transfection method with helper plasmids pAdΔF6 and pAAV2/9 (Penn Vector Core) as previously described [Wakimoto, H., et al., Curr. Protoc. Mol. Biol. 115: 23-16 (2016), incorporated herein by reference]. All constructs were administered at a titer of 5×10¹⁰ virus genome (vg)/kg via thoracic cavity injection to 7-week-old mice.

### Heart Weight Measurement and Immunofluorescence

Mice were anesthetised by isoflurane inhalation. The heart was arrested in diastole by injecting 500 μl of a 15% potassium chloride solution into the inferior vena cava. Hearts were excised and flushed with saline solution via retrograde perfusion. Aorta and auricles were trimmed, and hearts were dried by removing excess fluid with forceps. Heart weight was measured, after which hearts were immersed in 4% buffered formalin and embedded in paraffin blocks according to standard procedures.

5 μM sections were co-stained with cardiac troponin I (cTnl; Abcam, ab56357) and biotinylated wheat germ agglutinin (WGA; Vectorlabs, B-1025) with streptavidin-linked Alexa Fluor™ 488 conjugate (Life Technologies). Images were analysed using the NIKON-NIS-Elements Viewer software and ImageJ [Schneider, C. A., Rasband, W. S., and Eliceiri, K. W. Nat. Methods 9: 671-675 (2012), incorporated herein by reference]. For each heart, the diameters of 200 cardiomyocytes were counted and a mean value was obtained, with experimenters blinded to experimental condition until data collection was finalised.

Synthetic circmiR Generation

Based on established methods, the 3′ and 5′ group I permutated intron-exon (PIE) sequences [Umekage, S., and Kikuchi, Y. J. Biotechnol. 139: 265-272 (2009)] from the T4 phage were synthesised as gBlocks (IDT). These were inserted downstream of a T7 promoter in the pcDNA3.1 plasmid vector (Addgene). A miRNA sponge sequence carrying 12 bulged binding sites was cloned between these intron-exon sequences. To create a linear RNA control for comparison of exonuclease degradation susceptibility, the exon and 5′ half intron of the PIE sequence downstream the miRNA sponge sequence was removed by restriction enzyme digestion during plasmid linearisation prior to in vitro transcription. Both circular and linear constructs were then synthesised by in vitro transcription from linearised plasmid DNA template using TranscriptAid T7 High Yield Transcription Kit (Thermo Fisher Scientific). RNA was purified from the reactions using TRIzol™ LS reagent (Thermo Fisher Scientific). To confirm circularisation, 1 μg of RNA was digested with 2 U RNase R (Epicentre) at 37° C. for 30 minutes. Digests were separated on 2% agarose gels and bands were visualised using the ChemiDoc™ Imaging System (BioRad). Molar concentrations of synthetic circmiR were calculated using the NEBioCalculator™ tool (nebiocalculatorDOTnebDOTcom/#!/ssrnaamt).

### RNA Stability Assay

1.2×10⁵ HEK293T cells per well were seeded on 24-well culture plates. 0.25 μg of plasmids driving either circmiR or linear sponge expression were transfected using jetPRIME™ transfection reagent (Polyplus Transfection). Alternatively, 0.5 μg of T7 synthesised circmiRs and linear sponge RNA constructs were transfected using Lipofectamine™ 2000 (Invitrogen). 48 hours post-transfection, cells were treated with 10 μg/mL actinomycin D (Sigma Aldrich) in fresh media. Cells were harvested at 0-, 6-, 12-, 24-, 48-, 72-hour time points post-actinomycin treatment. RNA was isolated and equal RNA quantities were subjected to reverse-transcriptase-PCR and qPCR as described above. Each RNA level was normalised against the 0-h time point to calculate log₂ fold change.

### Statistical Analysis

All results are presented as mean+standard error of the mean (SEM). Two-tailed, unpaired Student's t-test was performed for comparison between two groups. One-way ANOVA followed by the Benjamini-Hochberg adjustment was used to compare more than two groups. All tests were performed using GraphPad Prism 7 Software. P<0.05 was considered significant.

### Example 2

Design and Circularisation of miRNA Sponge Sequences

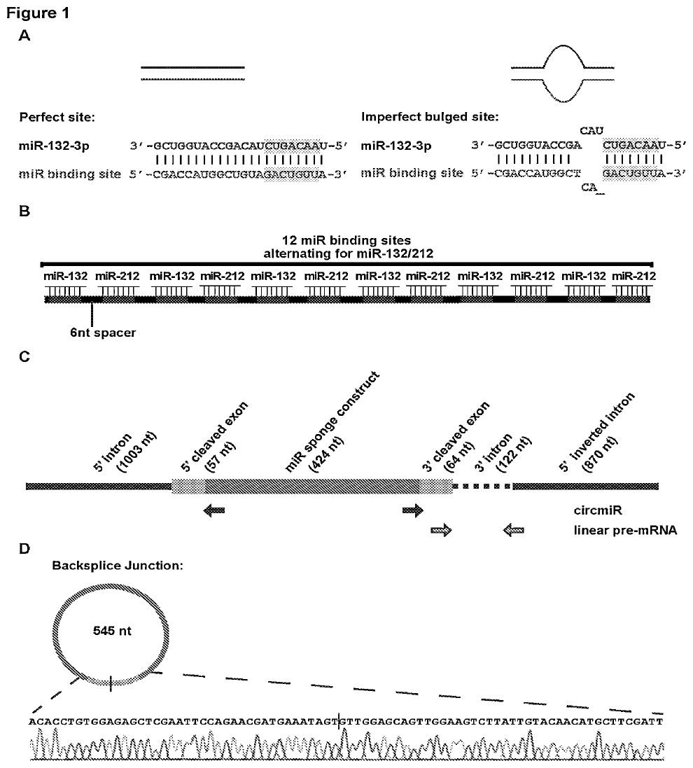

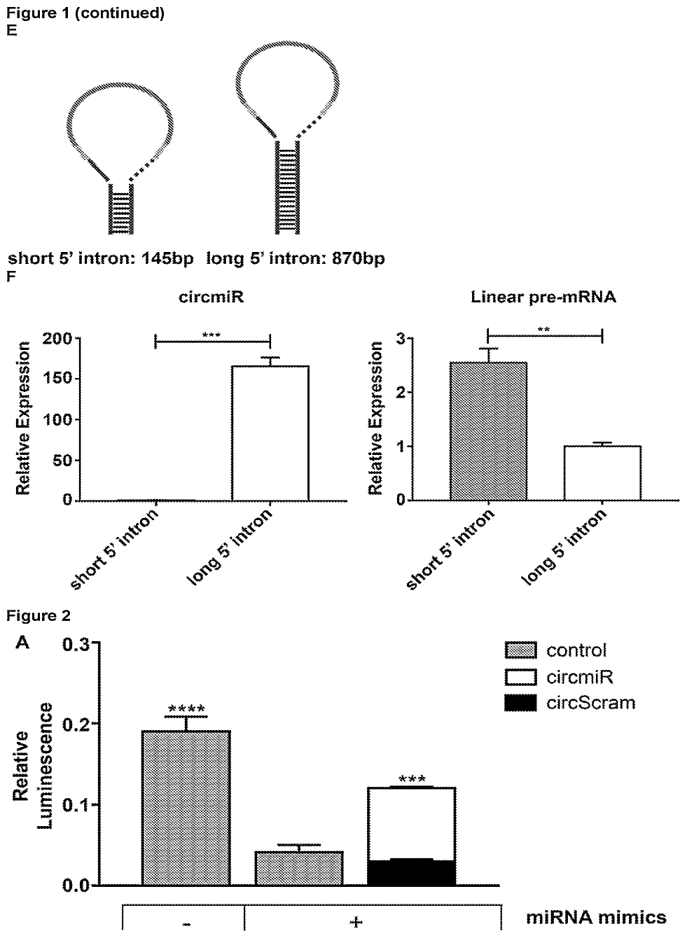

miRNA binding sites (MBS) were engineered as illustrated in FIG. 1A, and bulged binding sites were introduced by one deletion and two mismatches in the MBS (FIG. 1A). The sequences of the target miRNA and the sponge binding sites are shown in Table 4.

TABLE 4
sequences of miR-132 and miR-212 binding sites

| | | SEQ |
|---|---|---|
| | | ID |
| miRNA | Sequence | NO. |
| miR-132 | 3′-GCUGGUACCGACAUCUGACAAU-5′ | 1 |
| miR-132 bulged | 5′- CGACCAUGGCUCAGACUGUUA -3′ | 3 |
| binding site | | |
| miR-212 | 3′-ACCGGCACUGACCUCUGACAAU-5′ | 2 |
| miR-212 bulged | 5′-UGGCCGUGACUCCGACUGUUA-3′ | 4 |
| binding site | | |

The miRNA sponge was designed initially to carry a total of 12 alternating bulged miRNA binding sites, 6 for each of miR-132 and miR-212, with a 6 nucleotide (nt) separation space between miRNA binding sites (FIG. 1B). The values for these parameters were based on optimisation studies previously carried out for linear miRNA sponge design [Ebert, M. S., and Sharp, P. A. RNA 16: 2043-2050 (2010); Otaegi, G., et al., Front. Neurosci. 5: 146 (2012)].

To circularise the miRNA sponge, the sequence was flanked with inverted complementary introns (FIG. 10). The head-to-tail junction of the consequent circmiR was detected by quantitative real-time polymerase chain reaction (qPOR) using divergent primers and confirmed by Sanger sequencing (FIG. 1D). To test whether shorter flanking intronic sequences could also lead to efficient circularisation, the intronic sequences were trimmed (FIG. 1E). However, a significant decrease in circmiR and increase in linear pre-mRNA levels was observed, suggesting lower circularisation efficiency with shorter flanking introns (FIG. 1F). Hence, the construct with long inverted introns was used for the rest of the study.

### Example 3

Functional Efficacy Testing and Optimisation of circmiR Design In Vitro

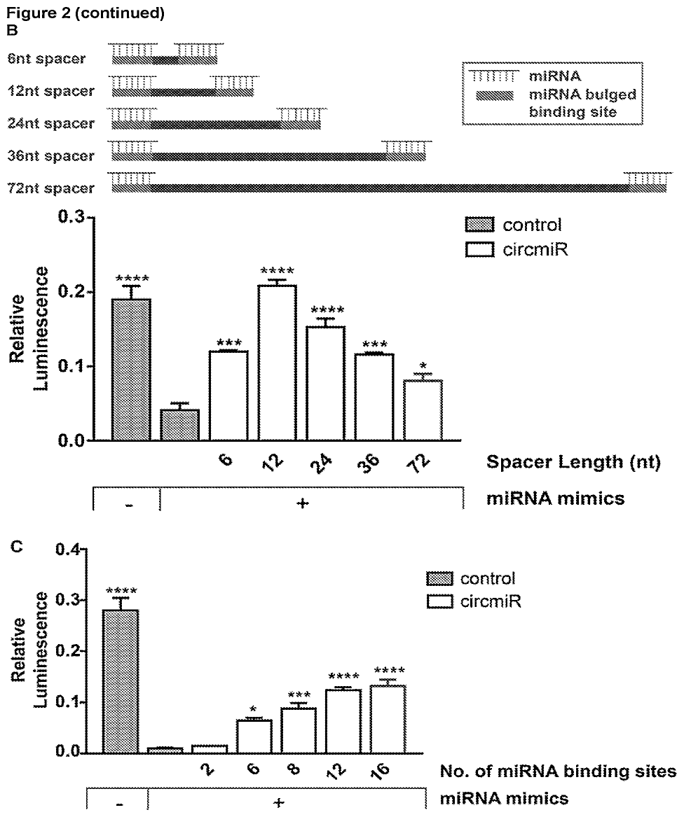

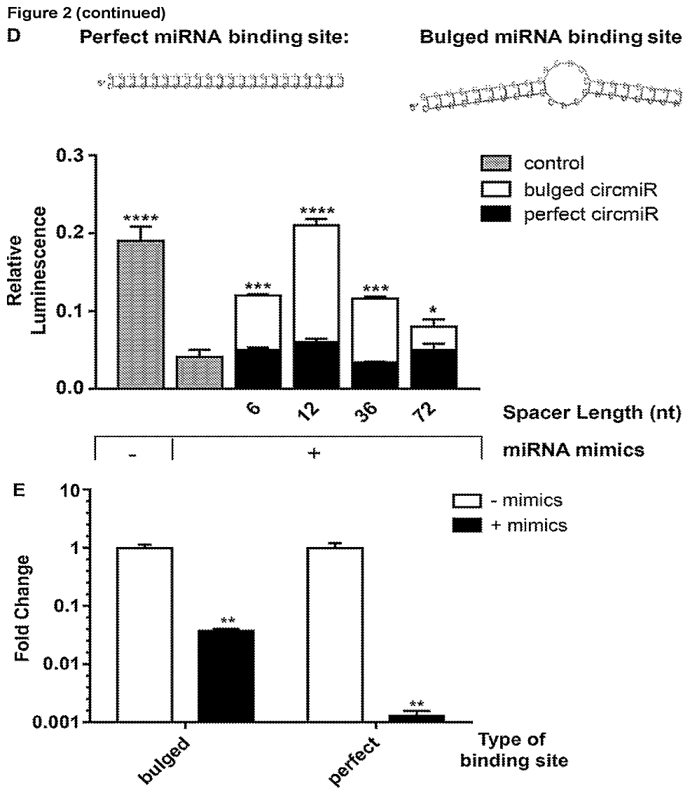

First, to validate circmiR function, miR-132 and -212 binding sites were inserted into the 3′-untranslated region (UTR) of a Renilla luciferase construct from the dual-luciferase reporter system. Co-transfecting this construct with miR-132 and miR-212 mimics into HEK293T cells caused a significant reduction in Renilla activity, as expected (FIG. 2A). Upon introduction of circmiR carrying bulged miRNA binding sites, Renilla activity was significantly rescued, compared to introduction of a negative control sponge (circScram), in which miR binding sites were scrambled (FIG. 2A).

The circmiR structural design was next optimised by testing the effect of different spacer lengths, type of binding sites and total number of binding sites. Previously, linear miRNA sponges showed effective miRNA inhibition with short spacers between miRNA binding sites [Otaegi, G., et al., Front. Neurosci. 5: 146 (2012)]. However, short spacer sequences in a circular structure may conceivably exert tension on neighbouring binding sites, affecting miRNA binding. To examine whether longer spacer lengths are preferable, bulged circmiRs with different spacer lengths were constructed: 6, 12, 24, 36, 72 nt. The 12-nt spacer construct produced the greatest rescue effect (FIG. 2B). Spacer sizes greater than 12-nt showed reduced rescue of Renilla activity (FIG. 2B). Notably, the 6-nt and 36-nt spacer constructs showed similar rescue effects (FIG. 2B).

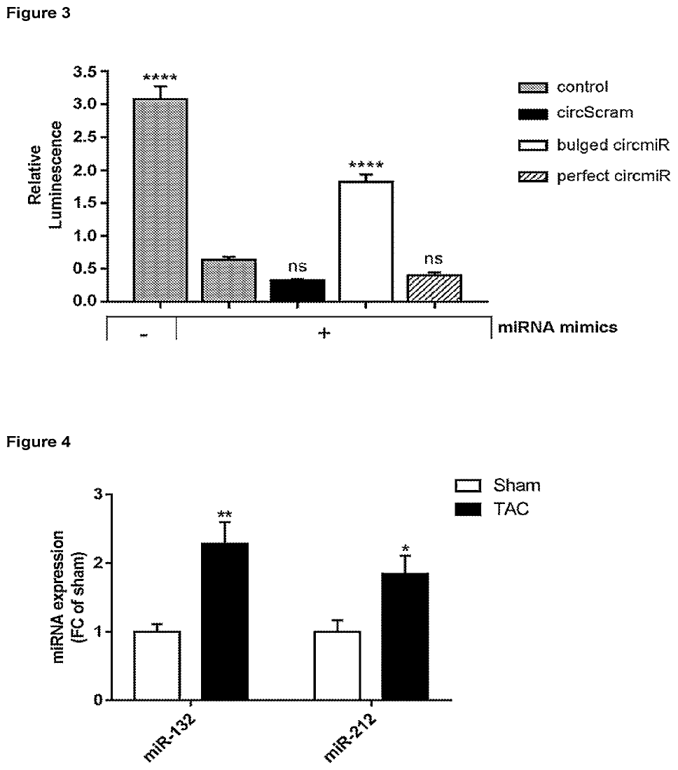

Bulged circmiR constructs were also generated containing 2, 6, 8, 12 and 16 binding sites, separated by 12-nt spacers, per sponge. The rescue effect of circmiR increased with increasing number of binding sites, but no significant difference was seen between circmiRs containing 12 and 16 binding sites (FIG. 2C). A bulged circmiR comprising 6 copies of SEQ ID NO: 3 alternating with 6 copies of SEQ ID NO: 4, and 2 copies of spacers SEQ ID NO: 27, 28, 29, 30 and 31 and 1 copy of spacer SEQ ID NO: 32 is set forth in SEQ ID NO: 13. Bulged miRNA binding sites are preferred over perfect binding sites in linear sponge sequences since the latter result in degradation via RISC-mediated endonucleolytic cleavage upon miRNA binding [Otaegi, G., et al., Front. Neurosci. 5: 146 (2012); Gentner, B., et al., Nat. Methods 6: 63-66 (2009)]. To determine if circmiRs suffer the same fate, we compared circmiRs with either bulged or perfect miRNA binding sites. The full nucleotide sequence of a circular RNA sponge comprising 6 perfect miR-132-3p and 6 perfect miR-212-3p binding sites, 12 nt random sequence spacers and slc8al exon 2 flanking sequences is set forth in SEQ ID NO: 16. Regardless of the spacer size, expression of perfect circmiRs failed to rescue Renilla activity (FIG. 2D). However, bulged circmiRs rescued Renilla expression consistently, and this effect was influenced by their spacer sizes as before (FIG. 2D). Interestingly, upon miRNA mimic treatment, both bulged and perfect circmiRs carrying 12 miRNA binding sites separated by 12-nt spacers were degraded (FIG. 2E). However, the perfect circmiRs were degraded to a much greater extent than bulged circmiRs (785-fold and 27-fold respectively; FIG. 2E). These luciferase experiments were replicated in H9C2 rat cardiomyocytes where luciferase activity was similarly rescued only by bulged but not perfect circmiRs, each carrying 12 miRNA binding sites separated by 12-nt spacers (FIG. 3).

Altogether, we concluded an optimal circmiR design, comprising of 12-nt spacers with 12 bulged miRNA binding sites. This design was used for all further experiments.

### Example 4

In Vivo circmiR Administration Attenuates Disease in a Mouse TAC Model

Hypertrophic stimuli reportedly upregulate cardiomyocyte expression of miR-132 and miR-212, which are necessary to drive pathological hypertrophy [Ucar, A., et al., Nat. Commun. 3: 1078 (2012)]. We confirmed the upregulation of both miR-132 and miR-212 in our in vivo transverse aortic constriction (TAC) cardiac pressure overload mouse model, although at a later timepoint than previously reported (FIG. 4).

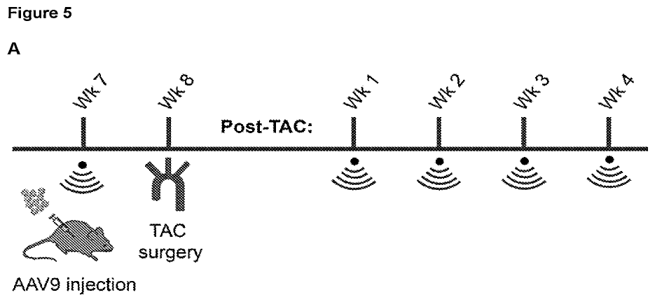

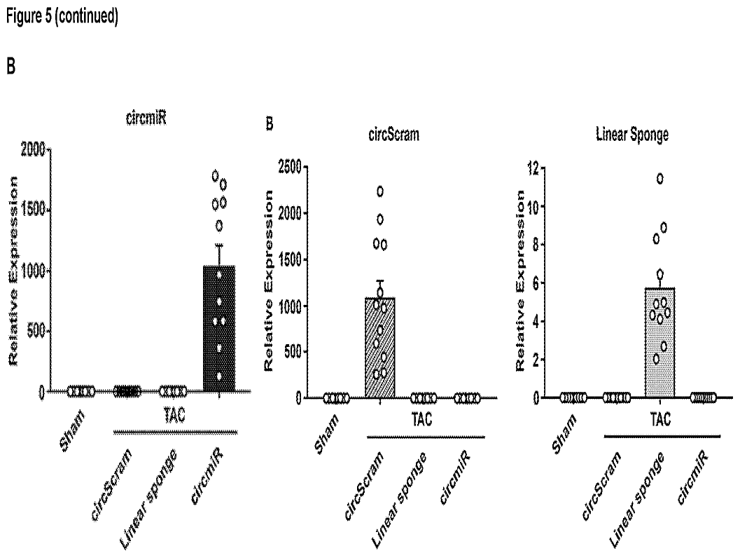

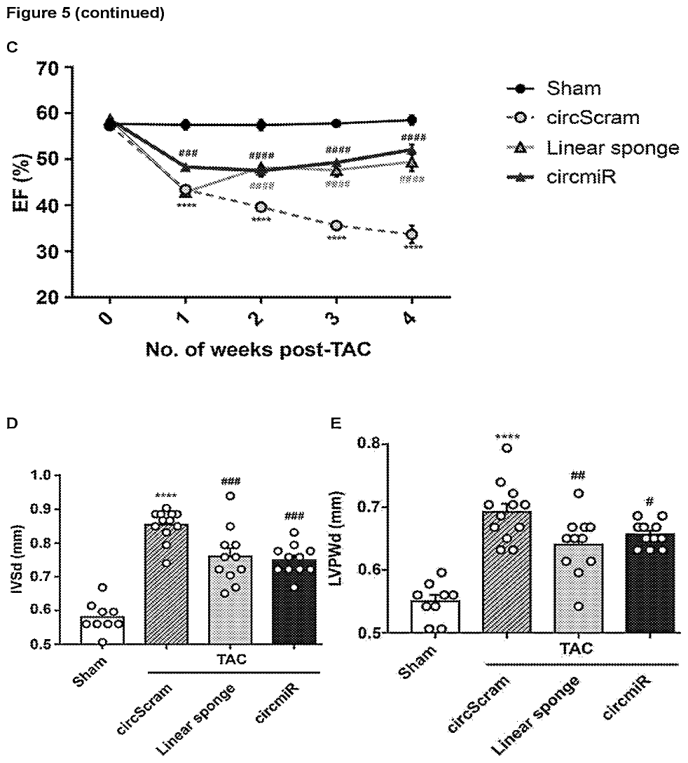

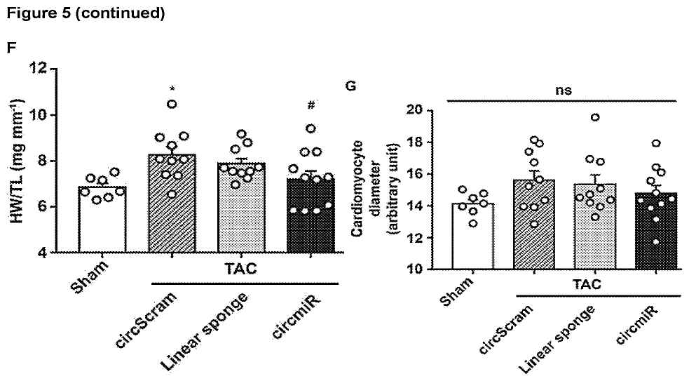

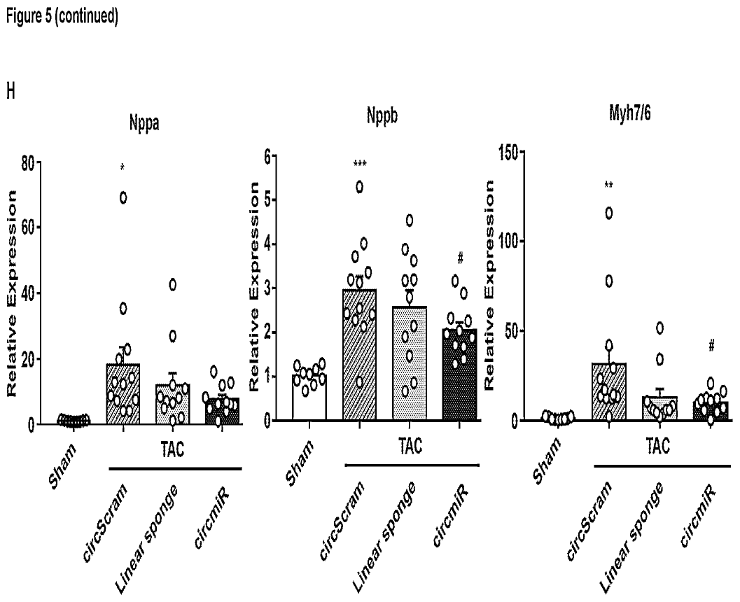

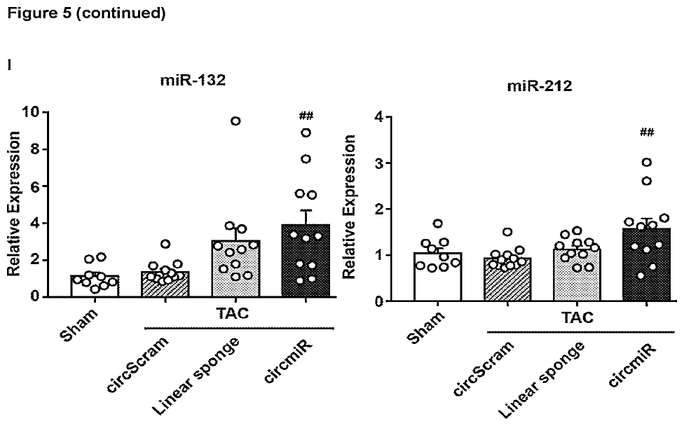

Next, adeno-associated virus (AAV) serotype 9 vectors were employed to deliver and express constructs in vivo, specifically in cardiomyocytes (CMs), using the cardiac troponin T (cTnT) promoter [Jiang, J., et al., Science 342: 111-114 (2013)]. AAV vectors expressing either circScram, circmiR or a linear miR-212/132 sponge (linsp) were injected intra-peritoneally one week before TAC surgery (FIG. 5A). Successful targeted expression was validated by qPCR analysis of isolated CMs (FIG. 5B). Specific divergent primers detected circScram and circmiR constructs respectively, whereas convergent primers detected linsp constructs, as expected (FIG. 5B and FIG. 6).

Cardiac function was evaluated by weekly echocardiographic measurements. Ejection fraction, a parameter of systolic cardiac function, was significantly compromised in TAC-operated circScram mice, whereas both circmiR and linsp mouse groups showed improved preservation of cardiac function up to 4 weeks post-TAC (FIG. 5C). Pressure overload induces pathological cardiac wall thickening (hypertrophy) [Anversa, P., et al., J. Am. Coll. Cardiol. 7: 1140-1149 (1986)]. 4 weeks post-TAC, interventricular septal (IVS) and left ventricular posterior wall (LVPVV) thickness of both circmiR and linear sponge groups were significantly reduced, compared to circScram (FIG. 5D, 5E). Thus, pathological hypertrophy was significantly attenuated in the circmiR and linear sponge treated groups, compared to circScram controls.

The heart weight to tibia length ratio, another measure of cardiac hypertrophy, increased post-TAC in both circScram and linsp mice, whereas heart weight in circmiR mice remained similar to sham levels (FIG. 5F). Morphologically, CM diameter trended to increase in both circScram and linsp mice more so than circmiR mice, in which CM diameter was again similar to those of sham mice, although these changes did not reach statistical significance (FIG. 5G).

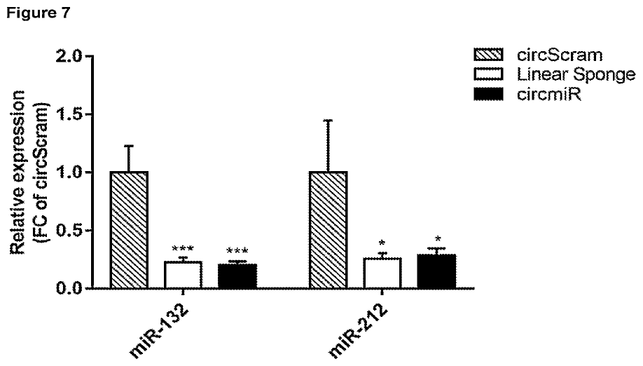

The expression levels of canonical CM stress markers Nppa, Nppb and Myh7 were significantly increased post-TAC in circScram-treated hearts compared to sham levels (FIG. 5H). These stress markers were attenuated in circmiR-treated hearts, for which decreases in Nppb and Myh7 were statistically significant (FIG. 5H). Attenuation of stress markers was also observed in linsp-treated hearts but did not reach statistical significance (FIG. 5H). Interestingly, the detected abundance of miR-132 and -212 were markedly higher in both circmiR and linsp groups compared to circScram controls (FIG. 5I). Conceivably, this may be caused by retention of sequestered miRNAs without degradation, due, in part, to bulged binding sites (FIG. 2E). Both circmiR and linsp did conversely appear to reduce miR-212/132 abundance in HEK293T cells in vitro (FIG. 7), possibly reflecting the considerable differences between cell types, and in vitro versus in vivo environments.

### Example 5

CircmiRs are More Stable than Linear miRNA Sponges

The stability of circmiRs in comparison to linear miRNA sponge constructs was investigated. HEK293T cells were transfected with plasmids driving either circmiR or linear sponge expression, or with synthetically generated circmiR or linear sponge constructs. Transcription was inhibited with actinomycin D and total RNA was harvested at indicated time points.

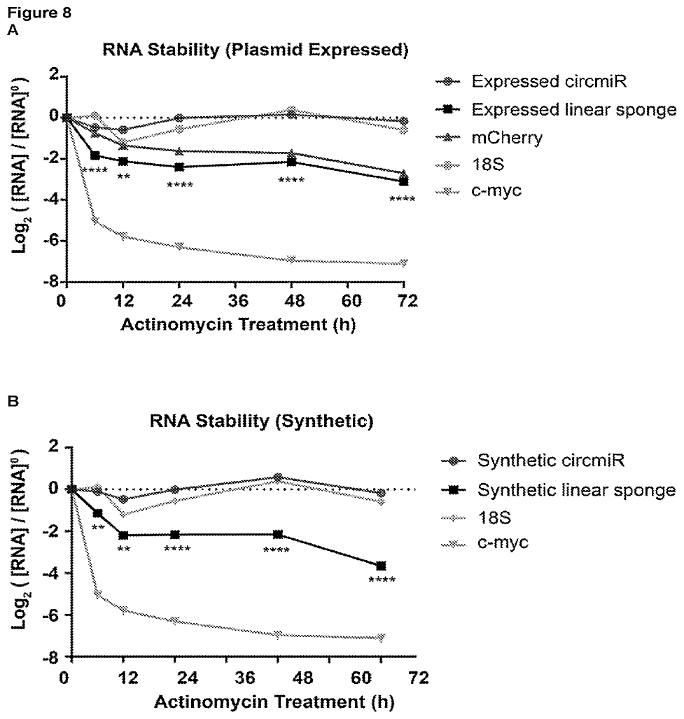

As anticipated [Jeck, W. R., et al., RNA 19: 141-157 (2013); Meganck, R. M., et al., Mol. Ther. Nucleic Acids 13: 89-98 (2018)], measured by qPCR, positive control 18S abundance was highly stable across the 72 h time period, whereas a decrease in abundance of the less stable c-Myc transcript (MYC) was observed, exhibiting a half-life of 6 h. Plasmid-expressed circmiRs were resistant to nuclease degradation and were stable for up to 72 h (FIG. 8A). In contrast, the turnover rate of the linear sponge RNA was comparable to that of an mCherry reporter (half-lives <24 h), also expressed from the same construct. Similar to plasmid-expressed circmiR, the synthetic circmiR was stable for up to 72 h, compared to synthetic linsp RNA (half-life <24 h) (FIG. 8B). Thus, these results strongly demonstrate that regardless of the source of circmiR production, plasmid-driven or synthetically produced, circular constructs are more stable than their linear counterparts.

### Example 6

Examples 1 to 3 demonstrated that the optimal design best suited for custom circmiRs targeting miR-212/132 comprised of a total of 12 alternate miRNA binding sites (6 for each miRNA) separated by a 12-nucleotide (nt) spacer. Comparing binding site type, bulged binding sites with an imperfect complementarity to the target miRNAs were effective whereas perfect binding sites failed to show any miRNA inhibitory effect in the luciferase assay.

Here, we extended our circmiR design to target 2 different sets of miRNAs, namely, miR-17-5p and miR-18a-5p (circmiR 1) as well as miR-20b-5p and miR-106a-5p (circmiR 2) (see Tables 5 and 6).

TABLE 5
Sequence of each of the native miRNA

| miRNA | miRBase ID | 5′ - Mature miRNA sequence - 3′ | SEQ ID NO. |
|---|---|---|---|
| hsa-miR-17-5p | MIMAT0000070 | CAAAGUGCUUACAGUGCAGGUAG | 5 |
| hsa-miR-18a-5p | MIMAT0000072 | UAAGGUGCAUCUAGUGCAGAUAG | 6 |
| hsa-miR-20b-5p | MIMAT0001413 | CAAAGUGCUCAUAGUGCAGGUAG | 9 |
| hsa-miR-106a-5p | MIMAT0000103 | AAAAGUGCUUACAGUGCAGGUAG | 10 |

TABLE 6
Sequence of each of the bulged and perfect miRNA binding sites

| miRNA | 5′ - miR binding site - 3′ | SEQ ID NO. |
|---|---|---|
| hsa-miR-17-5p perfect | CUACCUGCACUGUAAGCACUUUG | 49 |
| hsa-miR-17-5p bulged | CUACCUGCACUGAUGCACUUUG | 7 |
| hsa-miR-18a-5p perfect | CUAUCUGCACUAGAUGCACCUUA | 50 |
| hsa-miR-18a-5p bulged | CUAUCUGCACUACUGCACCUUA | 8 |
| hsa-miR-20b-5p perfect | CUACCUGCACUAUGAGCACUUUG | 51 |
| hsa-miR-20b-5p bulged | CUACCUGCACUAACGCACUUUG | 11 |
| hsa-miR-106a-5p perfect | CUACCUGCACUGUAAGCACUUUU | 52 |
| hsa-miR-106a-5p bulged | CUACCUGCACUGAUGCACUUUU | 12 |

As per the previous example, the binding sites for these miRNAs were alternated to reduce the frequency of recombination events that interferes with techniques used to construct the sponge such as PCR, cloning and Sanger sequencing.

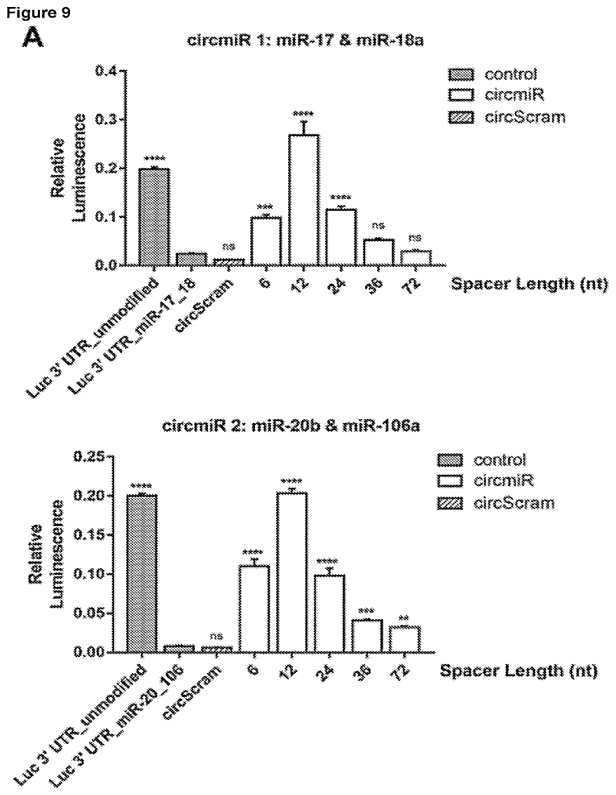

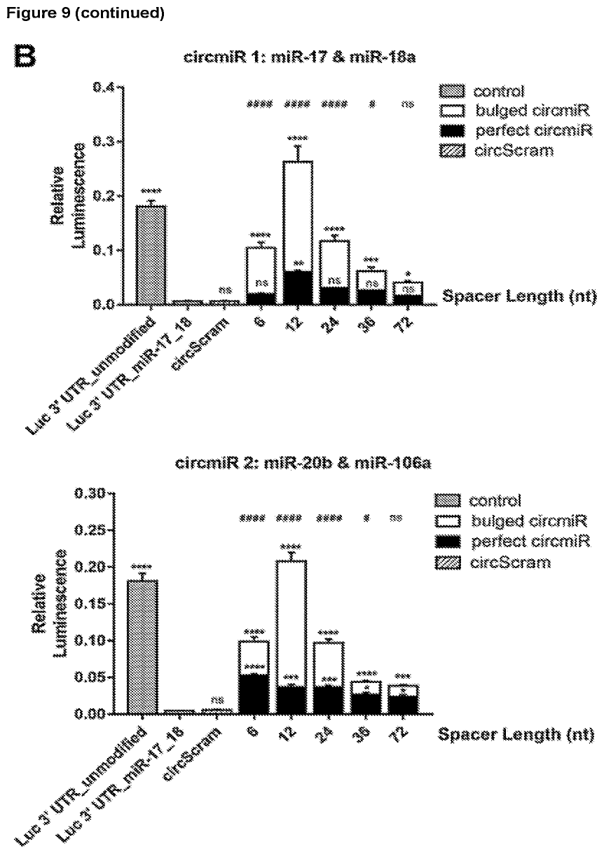

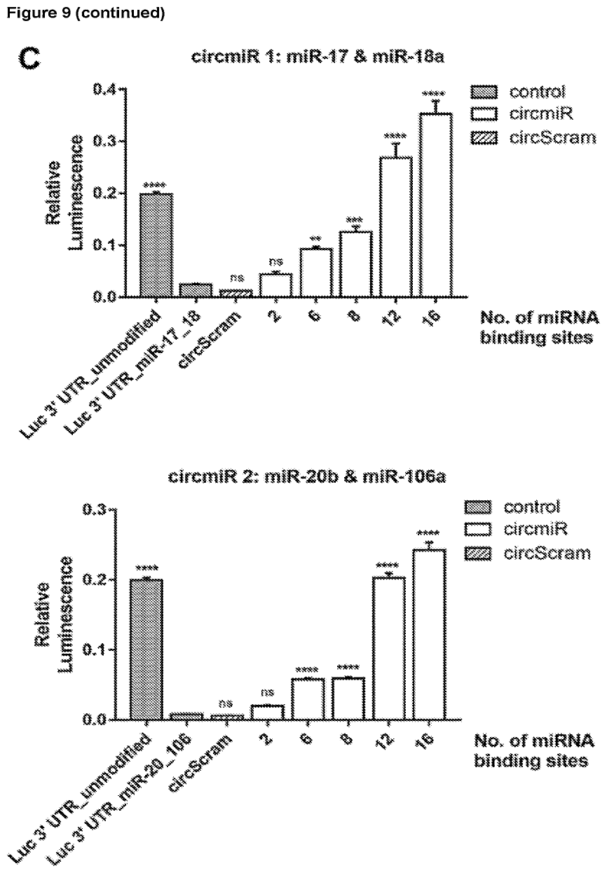

Either miR-17-5p and miR-18a-5p or miR-20b-5p and miR-106a-5p binding sites were inserted into the 3′-UTR of a Renilla luciferase construct from the dual-luciferase reporter system. It is worthwhile noting that these miRNAs have high endogenous expression within HEK293T cells that removes the need for including miRNA mimics in the experiment. Transfecting this construct, Luc 3′ UTR_miR, into HEK293T cells resulted in significant reduction in Renilla activity (FIG. 9). Upon introduction of circmiRs 1 and 2, Renilla activity was significantly rescued compared to circScram, a negative control sponge (FIG. 9).

To examine whether the same spacer length requirements as before were optimal for circmiRs 1 and 2, 12 bulged binding sites were separated by different spacer lengths: 6, 12, 24, 36, 72-nt. Both circmiRs 1 and 2 exerted the largest rescue effect with a 12-nt spacing consistently as before (FIG. 9A).

The type of binding site to be included was also tested by comparing circmiRs with bulged or perfect binding sites. Across different spacer lengths, bulged sites were more effective in rescuing Renilla activity compared to perfect sites (FIG. 9B). An exception to this was circmiR 1 carrying a 72-nt spacer length at which no significant difference was observed between bulged and perfect binding sites (FIG. 9B). This discrepancy could be accounted for by the less effective 72-nt spacer length at which efficacy between bulged and perfect binding sites may not be detected.

Bulged circmiR constructs were generated to contain 2, 6, 8, 12 and 16 binding sites, separated by 12-nt spacers, per sponge. The rescue effect of circmiR 1 increased with increasing number of binding sites as expected (FIG. 9C). While the rescue effect of circmiR 2 increased with increasing number of binding sites, both 6 and 8 binding sites had the same effect (FIG. 9C). While 16 binding sites were more effective than 12 binding sites for both circmiRs, 12 binding sites would be preferred to reduce the number of repeats incorporated into the construct. Moreover, both circmiRs 1 and 2 with 12 sites were able to rescue the luciferase activity to an extent greater than the control Luc 3′ UTR_unmodified.

The full nucleotide sequence of a circular RNA sponge comprising 6 bulged miR-17-5p alternating with 6 bulged miR-18a-5p binding sites, and 2 copies of 12 nt spacers SEQ ID NO: 27, 28, 29, 30 and 31 and 1 copy of spacer SEQ ID NO: 32 and slc8a1 exon 2 flanking sequences is set forth in SEQ ID NO: 14. The full nucleotide sequence of a circular RNA sponge comprising 6 bulged miR-20b-5p alternating with 6 bulged miR-106a-5p binding sites, and 2 copies of spacers SEQ ID NO: 27, 28, 29, 30 and 31 and 1 copy of spacer SEQ ID NO: 32 and slc8a1 exon 2 flanking sequences is set forth in SEQ ID NO: 15. The full nucleotide sequence of a circular RNA sponge comprising 6 perfect miR-17-5p and 6 perfect miR-18a-5p binding sites, 12 nt random sequence spacers and slc8a1 exon 2 flanking sequences is set forth in SEQ ID NO: 17. The full nucleotide sequence of a circular RNA sponge comprising 6 perfect miR-20b-5p and 6 perfect miR-106a-5p binding sites, 12 nt random sequence spacers and slc8a1 exon 2 flanking sequences is set forth in SEQ ID NO: 18.

Altogether, three pairs of miRNA binding sites were engineered into circmiR sponges and tested to determine optimum performance features, with the result that an optimal circmiR design would comprise 12 bulged miRNA binding sites with 12-nt spacers having random, non-identical sequences.

### SUMMARY

Compared to other anti-miR technology implemented to date, circmiRs, without the requirement for chemical modifications, are likely to be well tolerated in biological systems. The present invention demonstrates the successful construction of three different improved artificial circmiR sponges as miR-132 and miR-212; miR-17-5p and miR-18a-5p; and miR-20b-5p and miR-106a-5p antagonists and in vivo testing of artificial circmiRs targeting miR-132 and miR-212 in a cardiopathy model.

In our study, we used long flanking introns to maximise circularisation efficiency. However, if the circmiR design needs to be more compact due to viral vector space constraints, shorter flanking introns could be incorporated.

Alternating binding sites for any two miRNA pairs and including non-identical spacers reduces consecutive repeat sequences undergo recombination events. Recombination within the sponge sequence would make circmiRs difficult to construct that is not ideal. Importantly, we found that circmiRs constructed with 12 perfect sites showed poor miRNA inhibitory effect compared to bulged circmiRs. This could be due to the susceptibility of circmiRs containing perfect binding sites to degradation upon miRNA binding, by Ago2-mediated cleavage, or by RISC mediated endonucleolytic cleavage. The differences in spatial and structural distribution have varying effects on circmiR functionality. Hence, circmiR design plays utmost importance to its resulting structure that in turn determines its effectiveness as a miRNA inhibitor.

In a recent study describing the synthesis of a circular sponge comprising of 8 miRNA binding sites, both bulged or perfect binding sites were equally effective for miRNA inhibition [Jost, I., et al., RNA Biol. 15: 1032-1039 (2018)]. This discrepancy could be accounted for by the shorter post-transfection (4 hours) luciferase activity measurement during which susceptibility of the bulged or perfect binding sites to degradation may not be detected.

In the in vivo study presented herein, both circmiR and linsp, bearing binding sites for miR-132 and miR-212, attenuated cardiac hypertrophy and heart failure progression to a similar extent. This effect concurs with a previous study in which the pharmacologic inhibition of miR-132 by antagomir injection suppressed pressure-overload induced hypertrophy [Ucar, A., et al. Nat. Commun. 3: 1078 (2012)]. While ideal as proof-of-concept, cardiac AAV vector systems drive high levels of constant expression [Goncalves, M. A. (2005) Virol. J. 2, 43; McCarty, D. M., Young, S. M., and Samulski, R. J. (2004) Annu. Rev. Genet. 38, 819-845], which therefore likely preclude the benefit of the higher stability circmiR compared to its linear counterpart. However, therapeutically, a pharmacological agent that is more precisely and temporally dose-controlled is typically more desirable. It is in this context that we propose that the markedly improved stability of a directly administered synthetic circmiR may have the advantage against linear therapeutics.

Thus far, few publications have described the in vitro synthesis of circular miRNA sponges. A circular sponge that inhibits miR-21 in gastric carcinoma cells, and another that inhibits miR-122 from Hepatitis C Virus, have been reported to function in vitro [Jost, I., et al., (2018) RNA Biol. 15: 1032-1039; Liu, X., et al. (2018) Mol. Ther. Nucleic Acids 13: 312-321]. Notably, both studies employed enzymatic ligation of linear RNA generated from in vitro transcription as the method of RNA circularisation. We and others have recently shown efficacy of expressing endogenous circRNAs in mammalian models of disease [Lim, T. S. B., et al., (2019) Cardiovasc. Res. 115: 1998-2007; Shen, S. Y., et al. (2019) Ann Rheum Dis 78: 826-836]. In the present study, we utilised endogenous splice machinery to generate custom-designed circular miRNA sponges and delivered them into an in vivo mouse model using AAVs.

Modified miRNA inhibitors are currently the gold standard in clinical trials. Biopharmaceutical companies have several miRNA inhibitors in clinical pipelines with chemistries tweaked in various ways. The key advantage of circRNAs that stands out in its development into a miRNA sponge is that without any chemical modifications, these molecules are resistant to nuclease degradation that makes them more stable than linear RNAs. CircmiR half-lives are naturally longer than linear miRNA inhibitors, which would greatly reduce the cost of innovation in terms of having to tweak the aforementioned RNA chemistries. Furthermore, circmiRs are biodegradable, the mechanism for which still remains to be discovered, reducing the risk of plausible long-term toxicity with modified oligonucleotide chemistries.

Our findings demonstrate the promising potential of circmiRs as therapeutic miRNA antagonists. We have applied this treatment and demonstrate benefits in an in vivo model of cardiovascular disease, which represents the primary cause of death globally, and anticipate that future development can expand this scope significantly.

### REFERENCES

Any listing or discussion of an apparently prior-published document in this specification should not necessarily be taken as an acknowledgement that such document is part of the state of the art or is common general knowledge.
Ackers-Johnson, M., Li, P. Y., Holmes, A. P., O'Brien, S. M., Pavlovic, D., and Foo, R. S. (2016) A Simplified, Langendorff-Free Method for Concomitant Isolation of Viable Cardiac Myocytes and Nonmyocytes From the Adult Mouse Heart. Circ. Res. 119, 909
Anversa, P., Ricci, R., and Olivetti, G. (1986) Quantitative structural analysis of the myocardium during physiological growth and induced cardiac hypertrophy—a review. J. Am. Coll. Cardiol. 7, 1140-1149.
Blachinsky, E., Marbach, I., Cohen, R., Grably, M. R., and Engelberg, D. (2004) Procedure for controlling number of repeats, orientation, and order during cloning of oligonucleotides. BioTechniques 36, 933.
Creemers, E. E., Wilde, A. A., and Pinto, Y. M. (2011) Heart failure: advances through genomics. Nat. Rev. Genet. 12, 357-362 McDermott, A. M., Heneghan, H. M., Miller, N., and Kerin, M. J. (2011) The Therapeutic Potential of MicroRNAs: Disease Modulators and Drug Targets. Pharm. Res. 28, 3016-3029.
Ebbesen, K. K., Kjems, J., and Hansen, T. B. (2016) Circular RNAs: Identification, biogenesis and function. Biochim. Biophys. Acta 1859, 163-168.
Ebert, M. S., and Sharp, P. A. (2010) MicroRNA sponges: Progress and possibilities. RNA 16, 2043-2050.
Foinquinos, A., Batkai, S., Genschel, C., Viereck, J., Rump, S., Gyöngyösi, M., Traxler, D., Riesenhuber, M., Spannbauer, A., Lukovic, D., et al. (2020) Preclinical development of a miR-132 inhibitor for heart failure treatment. Nat. Commun. 11, 1-10.
Gentner, B., Schira, G., Giustacchini, A., Amendola, M., Brown, B. D., Ponzoni, M., and Naldini, L. (2009) Stable knockdown of microRNA in vivo by lentiviral vectors. Nat. Methods 6, 63-66.
Goncalves, M. A. (2005) Adeno-associated virus: from defective virus to effective vector. Virol. J. 2, 43.
Hansen, T. B., Jensen, T. I., Clausen, B. H., Bramsen, J. B., Finsen, B., Damgaard, C. K., and Kjems, J. (2013) Natural RNA circles function as efficient microRNA sponges. Nature 495, 384-388.
Janssen, H. L. A., Reesink, H. W., Lawitz, E. J., Zeuzem, S., Rodriguez-Torres, M., Patel, K., van der Meer, A. J., Patick, A. K., Chen, A., Zhou, Y., et al. (2013) Treatment of HCV Infection by Targeting MicroRNA. N. Engl. J. Med. 368, 1685-1694.
Jeck, W. R., Sorrentino, J. A., Wang, K., Slevin, M. K., Burd, C. E., Liu, J. Z., Marzluff, W. F., and Sharpless, N. E. (2013) Circular RNAs are abundant, conserved, and associated with ALU repeats. RNA 19, 141-157.
Jiang, J., Wakimoto, H., Seidman, J. G., and Seidman, C. E. (2013) Allele-Specific Silencing of Mutant Myh6 Transcripts in Mice Suppresses Hypertrophic Cardiomyopathy. Science 342, 111-114.
Jost, I., Shalamova, L. A., Gerresheim, G. K., Niepmann, M., Bindereif, A., and Rossbach, O. (2018) Functional sequestration of microRNA-122 from Hepatitis C Virus by circular RNA sponges. RNA Biol. 15, 1032-1039.
Kruger, J., and Rehmsmeier, M. (2006) RNAhybrid: microRNA target prediction easy, fast and flexible. Nucleic Acids Res. 34, W451-W454.
Kumarswamy, R., Volkmann, I., Beermann, J., Napp, L. C., Jabs, O., Bhayadia, R., Melk, A., Ucar, A., Chowdhury, K., and Lorenzen, J. M. (2014) Vascular importance of the miR-212/132 cluster. European heart journal 35, 3224-3231.
Lim, T. S. B., Aliwarga, E., Luu, T. D. A., Li, Y. Q. P., Ng, S. L., Annadoray, L., Sian, S., Ackers-Johnson, M. A., and Foo, R. S. Y. (2019) Targeting the highly abundant circular RNA circS1c8a1 in cardiomyocytes attenuates pressure overload induced hypertrophy. Cardiovasc. Res. 115, 1998-2007.
Liu, X., Abraham, J. M., Cheng, Y. L., Wang, Z. X., Wang, Z., Zhang, G. J., Ashktorab, H., Smoot, D. T., Cole, R. N., Boronina, T. N., et al. (2018) Synthetic Circular RNA Functions as a miR-21 Sponge to Suppress Gastric Carcinoma Cell Proliferation. Mol. Ther. Nucleic Acids 13, 312-321.
Meganck, R. M., Borchardt, E. K., Rivera, R. M. C., Scalabrino, M. L., Wilusz, J. E., Marzluff, W. F., and Asokan, A. (2018) Tissue-Dependent Expression and Translation of Circular RNAs with Recombinant AAV Vectors In Vivo. Mol. Ther. Nucleic Acids 13, 89-98.
McCarty, D. M., Young, S. M., and Samulski, R. J. (2004) Integration of adeno-associated virus (AAV) and recombinant AAV vectors. Annu. Rev. Genet. 38, 819-845.
Otaegi, G., Pollock, A., and Sun, T. (2012) An optimized sponge for microRNA miR-9 affects spinal motor neuron development in vivo. Front. Neurosci. 5, 146.
Rehmsmeier, M., Steffen, P., Hochsmann, M., and Giegerich, R. (2004) Fast and effective prediction of microRNA/target duplexes. RNA 10, 1507-1517.
Rockman, H. A., Ross, R. S., Harris, A. N., Knowlton, K. U., Steinhelper, M. E., Field, L. J., Ross, J., and Chein, K. R. (1991) Segregation of atrial-specific and inducible expression of an atrial-natriuretic-factor transgene in an invivo murine model of cardiac-hypertrophy. Proc. Natl. Acad. Sci. U.S.A 88, 8277-8281.
Schneider, C. A., Rasband, W. S., and Eliceiri, K. W. (2012) NIH Image to ImageJ: 25 years of image analysis. Nat. Methods 9, 671-675.
Shen, S. Y., Wu, Y. Z., Chen, J. X., Xie, Z., Huang, K. M., Wang, G. L., Yang, Y. T., Ni, W. Y., Chen, Z. J., Shi, P. H., et al. (2019) CircSERPINE2 protects against osteoarthritis by targeting miR-1271 and ETS-related gene. Ann Rheum Dis 78, 826-836.
Tan, W. L. W., Lim, B. T. S., Anene-Nzelu, C. G. O., Ackers-Johnson, M., Dashi, A., See, K., Tiang, Z. N., Lee, D. P., Chua, W. W., Luu, T. D. A., et al. (2017) A landscape of circular RNA expression in the human heart. Cardiovasc. Res. 113, 298-309.
Ucar, A., Gupta, S. K., Fiedler, J., Erikci, E., Kardasinski, M., Batkai, S., Dangwal, S., Kumarswamy, R., Bang, C., Holzmann, A., et al. (2012) The miRNA-212/132 family regulates both cardiac hypertrophy and cardiomyocyte autophagy. Nat. Commun. 3, 1078.
Umekage, S., and Kikuchi, Y. (2009) In vitro and in vivo production and purification of circular RNA aptamer. J. Biotechnol. 139, 265-272.
Wakimoto, H., Seidman, J. G., Foo, R. S. Y., and Jiang, J. (2016) AAV9 Delivery of shRNA to the Mouse Heart. Curr. Protoc. Mol. Biol. 115, 23-16.

1. An isolated circular RNA polynucleotide microRNA sponge, comprising:
a) a plurality of human or non-human animal microRNA bulged binding sites, wherein each binding site comprises a region which is 100% complementary to a microRNA seed region;
b) a plurality of polynucleotide spacers, wherein each spacer is of 6 to 24 nucleotides and positioned between two binding sites;
wherein said plurality of spacers comprises at least two spacers having a random, non-identical, sequence.

2. The isolated circular RNA polynucleotide microRNA sponge of claim 1, comprising at least 3 of each of two different microRNA bulged binding sites, and/or
wherein said circular RNA polynucleotide contains a total of 12 microRNA binding sites, and/or
wherein said circular RNA polynucleotide contains spacers of 12 nucleotides in length between the microRNA binding sites, and/or
wherein said different microRNA binding sites are alternated in the circular RNA polynucleotide.

3. (canceled)

4. (canceled)

5. (canceled)

6. The isolated circular RNA polynucleotide according to claim 1, wherein each bulge is created by a one base deletion and two base mismatches at positions 9-11 nt from the 3′ end of each binding site.

7. The isolated circular RNA polynucleotide according to claim 1, comprising:
i) human or non-human animal miR-132 microRNA bulged binding sites and human or non-human animal miR-212 microRNA bulged binding sites;
ii) human or non-human animal miR-17-5p microRNA bulged binding sites and human or non-human animal miR-18a-5p microRNA bulged binding sites; or
iii) human or non-human animal miR-20b-5p microRNA bulged binding sites and human or non-human animal miR-106a-5p microRNA bulged binding sites,
wherein each binding site comprises a region which is 100% complementary to the microRNA seed region and is separated by a polynucleotide spacer of eight to twenty nucleotides which comprises a random, non-identical, sequence to reduce repetition of sequences within the sponge and wherein binding sites directed to different microRNA are alternated to reduce repetition of sequences within the sponge.

8. The isolated circular RNA polynucleotide according to claim 7, comprising twelve bulged binding sites selected from the group comprising, six miR-132 bulged binding sites alternating with six miR-212 bulged binding sites; six miR-17-5p bulged binding sites alternating with six miR-18a-5p bulged binding sites; and six miR-20b-5p bulged binding sites alternating with six miR-106a-5p bulged binding sites; and spacers of twelve nucleotides between each binding site.

9. The isolated circular RNA polynucleotide according to claim 7, wherein:
i) respective binding sites are complementary to the miR-132 microRNA nucleic acid sequence set forth in 3′-GCUGGUACCGACAUCUGACAAU-5′ (SEQ ID NO: 1) and complementary to the miR-212 microRNA nucleic acid sequence set forth in 3′-ACCGGCACUGACCUCUGACAAU-5′ (SEQ ID NO: 2); or
ii) respective binding sites are complementary to the miR-17-5p microRNA nucleic acid sequence set forth in 3′-CAAAGUGCUUACAGUGCAGGUAG-5′ (SEQ ID NO: 5) and complementary to the miR-18a-5p microRNA nucleic acid sequence set forth in 3′-UAAGGUGCAUCUAGUGCAGAUAG-5′ (SEQ ID NO: 61; or
iii) respective binding sites are complementary to the miR-20b-5p microRNA nucleic acid sequence set forth in 3′-CAAAGUGCUCAUAGUGCAGGUAG-5′ (SEQ ID NO: 9) and complementary to the miR-106a-5p microRNA nucleic acid sequence set forth in 3′-AAAAGUGCUUACAGUGCAGGUAG-5′ (SEQ ID NO: 10).

10. The isolated circular RNA polynucleotide according to claim 7, wherein:
i) the bulged binding site for miR-132 comprises the nucleic acid sequence set forth in 5′-CGACCAUGGCTCAGACUGUUA-3′ (SEQ ID NO: 3) and the bulged binding site for miR-212 comprises the nucleic acid sequence set forth in 5′-UGGCCGUGACUCCGACUGUUA-3′ (SEQ ID NO: 4);
ii) the bulged binding site for miR-17-5p comprises the nucleic acid sequence set forth in 3′-CTACCTGCACTGATGCACTTTG-5′ (SEQ ID NO: 7) and the bulged binding site for miR-18a-5p comprises the nucleic acid sequence set forth in 3′-CTATCTGCACTACTGCACCTTA-5′ (SEQ ID NO: 8); or
iii) the bulged binding site for miR-20b-5p comprises the nucleic acid sequence set forth in 3′-CTACCTGCACTAACGCACTTTG-5′ (SEQ ID NO: 11) and the bulged binding site for miR-106a-5p comprises the nucleic acid sequence set forth in 3′-CTACCTGCACTGATGCACTTTT-5′ (SEQ ID NO: 12).

11. The isolated circular RNA polynucleotide according to claim 10, comprising the nucleic acid sequence selected from the nucleic acid sequences set forth in the group comprising:
i) SEQ ID NO: 13, comprising 6 copies of SEQ ID NO: 3 alternating with 6 copies of SEQ ID NO: 4, and 2 copies of spacers SEQ ID NO: 27, 28, 29, 30 and 31 and 1 copy of spacer SEQ ID NO: 32;
ii) SEQ ID NO: 14, comprising 6 copies of SEQ ID NO: 7 alternating with 6 copies of SEQ ID NO: 8, and 2 copies of spacers SEQ ID NO: 27, 28, 29, 30 and 31 and 1 copy of spacer SEQ ID NO: 32; and
iii) SEQ ID NO: 15, comprising 6 copies of SEQ ID NO: 11 alternating with 6 copies of SEQ ID NO: 12, and 2 copies of spacers SEQ ID NO: 27, 28, 29, 30 and 31 and 1 copy of spacer SEQ ID NO: 32.

12. The isolated circular RNA polynucleotide according to claim 1, for use as a medicament.

13. The isolated circular RNA polynucleotide according to claim 12, for use in the treatment of:
a) cardiomyopathy, when the circular RNA polynucleotide comprises miR-132 bulged binding sites and miR-212 bulged binding sites; or
b) cancer, when the circular RNA polynucleotide comprises miR-17-5p bulged binding sites and miR-18a-5p bulged binding sites, or comprises miR-20b-5p bulged binding sites and miR-106a-5p bulged binding sites.

14. A pharmaceutical composition comprising a circular RNA polynucleotide of claim 1; and at least one of a pharmaceutically acceptable diluent, carrier and adjuvant.

15. An isolated DNA expression construct comprising a nucleic acid sequence encoding the circular RNA polynucleotide according to claim 1, operably linked to a promoter, inverted complementary introns flanking the RNA polynucleotide microRNA sponge sequence, a splice acceptor site (SA) and a splice donor site (SD).

16. The isolated DNA expression construct of claim 15, comprising a nucleic acid sequence encoding the isolated circular RNA polynucleotide comprising the nucleic acid sequence selected from the nucleic acid sequences set forth in the group comprising:
i) SEQ ID NO: 13, comprising 6 copies of SEQ ID NO: 3 alternating with 6 copies of SEQ ID NO: 4, and 2 copies of spacers SEQ ID NO: 27, 28, 29, 30 and 31 and 1 copy of spacer SEQ ID NO: 32;
ii) SEQ ID NO: 14, comprising 6 copies of SEQ ID NO: 7 alternating with 6 copies of SEQ ID NO: 8, and 2 copies of spacers SEQ ID NO: 27, 28, 29, 30 and 31 and 1 copy of spacer SEQ ID NO: 32; and
iii) SEQ ID NO: 15, comprising 6 copies of SEQ ID NO: 11 alternating with 6 copies of SEQ ID NO: 12, and 2 copies of spacers SEQ ID NO: 27, 28, 29, 30 and 31 and 1 copy of spacer SEQ ID NO: 32.

17. An expression vector comprising the DNA expression construct of claim 15.

18. The expression vector of claim 17, wherein the expression vector comprises a constitutive promoter or an inducible promoter, or a cardiac- or cardiomyocyte-specific promoter.

19. The expression vector of claim 18, wherein the promoter is selected from the group comprising a cardiac troponin T promoter (cTnT), an α-myosin heavy chain (α-MHC) promoter and a myosin light chain (MLC2v) promoter.

20. The expression vector of claim 17, wherein the expression vector is a virus expression vector selected from the group consisting of Lentivirus, Adenovirus and Adeno-associated virus (AAV).

21. An isolated circular RNA polynucleotide, pharmaceutical composition, expression construct or expression vector of claim 1 for the treatment, amelioration or prevention of a disease or medical disorder associated with the presence or over-expression of a plurality of microRNA.

22. The isolated circular RNA polynucleotide of claim 21, wherein the microRNA comprises
miR-132 and miR-212,
miR-17-5p and miR-18a-5p, or
miR-20b-5p and miR-106a-5p.

23. The isolated circular RNA polynucleotide of claim 21, wherein the disease or medical disorder is:
a) cardiomyopathy, when the circular RNA polynucleotide comprises miR-132 bulged binding sites and miR-212 bulged binding sites; or
b) cancer, when the circular RNA polynucleotide comprises miR-17-5p bulged binding sites and miR-18a-5p bulged binding sites, or comprises miR-20b-5p bulged binding sites and miR-106a-5p bulged binding sites.

24. (canceled)

25. (canceled)

26. (canceled)

27. A method for the treatment, amelioration or prevention of a disease or medical disorder associated with the presence or over-expression of a plurality of microRNA, comprising the step of administering an efficacious amount of a circular RNA polynucleotide of claim 1, a pharmaceutical composition comprising the circular RNA polynucleotide, an expression construct comprising a nucleic acid sequence encoding the circular RNA polynucleotide, or an expression vector comprising the expression construct to a human or non-human animal in need of such treatment.

28. The method of claim 27, wherein the microRNA comprises
miR-132 and miR-212,
miR-17-5p and miR-18a-5p, or
miR-20b-5p and miR-106a-5p.

29. The method of claim 27, wherein the disease or medical disorder is:
a) cardiomyopathy, when the circular RNA polynucleotide comprises miR-132 bulged binding sites and miR-212 bulged binding sites; or
b) cancer, when the circular RNA polynucleotide comprises miR-17-5p bulged binding sites and miR-18a-5p bulged binding sites, or comprises miR-20b-5p bulged binding sites and miR-106a-5p bulged binding sites.

30. A method of optimizing the structure of a circular RNA polynucleotide microRNA sponge comprising a plurality of bulged binding sites directed to human or non-human animal target miRNA, comprising the steps;
a) test the effect of a plurality of spacers of 6 to 24 nucleotides in length between binding sites, in a circular RNA polynucleotide microRNA sponge comprising a plurality of bulged binding sites directed to one or more human or non-human animal target miRNA, on the binding to their target miRNA, and select the optimum spacer length;
b) test the effect of at least 6 binding sites in total, in a circular RNA polynucleotide microRNA sponge comprising a plurality of bulged binding sites directed to one or more human or non-human animal target miRNA, on the binding to their target miRNA;
c) engineer a circular RNA polynucleotide microRNA sponge comprising the optimum spacer length and number of binding sites from a) and b),
wherein said plurality of spacers comprises at least two spacers having a random, non-identical, sequence.

31. The method of claim 30, wherein the sponge comprises alternating binding sites.

32. An isolated circular RNA polynucleotide microRNA sponge produced according to the method of claim 30.


SEQUENCE LISTING (52 sequences)

SEQ ID NO: 1 - RNA, 22 nt, Artificial Sequence
  miR-132
gcugguaccgacaucugacaau

SEQ ID NO: 2 - RNA, 22 nt, Artificial Sequence
  miR-212
accggcacugaccucugacaau

SEQ ID NO: 3 - RNA, 21 nt, Artificial Sequence
  miR-132 bulged binding site
cgaccauggcucagacuguua

SEQ ID NO: 4 - RNA, 21 nt, Artificial Sequence
  miR-212 bulged binding site
uggccgugacuccgacuguua

SEQ ID NO: 5 - RNA, 23 nt, Artificial Sequence
  miR-17-5p
caaagugcuuacagugcagguag

SEQ ID NO: 6 - RNA, 23 nt, Artificial Sequence
  miR-18a-5p
uaaggugcaucuagugcagauag

SEQ ID NO: 7 - RNA, 22 nt, Artificial Sequence
  miR-17-5p bulged binding site
cuaccugcacugaugcacuuug

SEQ ID NO: 8 - RNA, 22 nt, Artificial Sequence
  miR-18a-5p bulged binding site
cuaucugcacuacugcaccuua

SEQ ID NO: 9 - RNA, 23 nt, Artificial Sequence
  miR-20b-5p
caaagugcucauagugcagguag

SEQ ID NO: 10 - RNA, 23 nt, Artificial Sequence
  miR-106a-5p
aaaagugcuuacagugcagguag

SEQ ID NO: 11 - RNA, 22 nt, Artificial Sequence
  miR-20b-5p bulged binding site
cuaccugcacuaacgcacuuug

SEQ ID NO: 12 - RNA, 22 nt, Artificial Sequence
  miR-106a-5p bulged binding site
cuaccugcacugaugcacuuuu

SEQ ID NO: 13 - DNA, 545 bp, Artificial Sequence
  Full sequence of bulged circmR miR-132 and miR-212
gttggagcagttggaagtcttattgtacaacatgcttcgattaagtctcccacccaaccgcggggaagatcccgaccatggctcagactgttatcagcagctgtgtggccgtgactccgactgttaactcgctattagcgaccatggctcagactgttaactcctatcgtatggccgtgactccgactgttatcatgactaccgcgaccatggctcagactgttactataagaagcatggccgtgactccgactgttaattcggagatcccgaccatggctcagactgttatcagcagctgtgtggccgtgactccgactgttaactcgctattagcgaccatggctcagactgttaactcctatcgtatggccgtgactccgactgttatcatgactaccgcgaccatggctcagactgttactataagaagcatggccgtgactccgactgttaattcggagatctggatagcggccgcgaggtggaggggaagactttgaggacacctgtggagagctcgaattccagaacgatgaaatagt

SEQ ID NO: 14 - DNA, 557 bp, Artificial Sequence
  Full sequence of bulged circmiR 1 miR-17 and miR-18a
gttggagcagttggaagtcttattgtacaacatgcttcgattaagtctcccacccaaccgcggggaagatccctacctgcactgatgcactttgtcagcagctgtgctatctgcactactgcaccttaactcgctattagctacctgcactgatgcactttgactcctatcgtactatctgcactactgcaccttatcatgactaccgctacctgcactgatgcactttgctataagaagcactatctgcactactgcaccttaattcggagatccctacctgcactgatgcactttgtcagcagctgtgctatctgcactactgcaccttaactcgctattagctacctgcactgatgcactttgactcctatcgtactatctgcactactgcaccttatcatgactaccgctacctgcactgatgcactttgctataagaagcactatctgcactactgcaccttaattcggagatctggatagcggccgcgaggtggaggggaagactttgaggacacctgtggagagctcgaattccagaacgatgaaatagt

SEQ ID NO: 15 - DNA, 557 bp, Artificial Sequence
  Full sequence of bulged circmiR 2 miR-20b and miR-106a
gttggagcagttggaagtcttattgtacaacatgcttcgattaagtctcccacccaaccgcggggaagatccctacctgcactaacgcactttgtcagcagctgtgctacctgcactgatgcacttttactcgctattagctacctgcactaacgcactttgactcctatcgtactacctgcactgatgcactttttcatgactaccgctacctgcactaacgcactttgctataagaagcactacctgcactgatgcacttttattcggagatccctacctgcactaacgcactttgtcagcagctgtgctacctgcactgatgcacttttactcgctattagctacctgcactaacgcactttgactcctatcgtactacctgcactgatgcactttttcatgactaccgctacctgcactaacgcactttgctataagaagcactacctgcactgatgcacttttattcggagatctggatagcggccgcgaggtggaggggaagactttgaggacacctgtggagagctcgaattccagaacgatgaaatagt

SEQ ID NO: 16 - DNA, 557 bp, Artificial Sequence
  Full sequence of perfect circmR miR-132 and miR-212
gttggagcagttggaagtcttattgtacaacatgcttcgattaagtctcccacccaaccgcggggaagatcccgaccatggctgtagactgttatcagcagctgtgtggccgtgactggagactgttaactcgctattagcgaccatggctgtagactgttaactcctatcgtatggccgtgactggagactgttatcatgactaccgcgaccatggctgtagactgttactataagaagcatggccgtgactggagactgttaattcggagatcccgaccatggctgtagactgttatcagcagctgtgtggccgtgactggagactgttaactcgctattagcgaccatggctgtagactgttaactcctatcgtatggccgtgactggagactgttatcatgactaccgcgaccatggctgtagactgttactataagaagcatggccgtgactggagactgttaattcggagatctggatagcggccgcgaggtggaggggaagactttgaggacacctgtggagagctcgaattccagaacgatgaaatagt

SEQ ID NO: 17 - DNA, 569 bp, Artificial Sequence
  Full sequence of perfect circmR1 miR-17 and miR-18a
gttggagcagttggaagtcttattgtacaacatgcttcgattaagtctcccacccaaccgcggggaagatccctacctgcactgtaagcactttgtcagcagctgtgctatctgcactagatgcaccttaactcgctattagctacctgcactgtaagcactttgactcctatcgtactatctgcactagatgcaccttatcatgactaccgctacctgcactgtaagcactttgctataagaagcactatctgcactagatgcaccttaattcggagatccctacctgcactgtaagcactttgtcagcagctgtgctatctgcactagatgcaccttaactcgctattagctacctgcactgtaagcactttgactcctatcgtactatctgcactagatgcaccttatcatgactaccgctacctgcactgtaagcactttgctataagaagcactatctgcactagatgcaccttaattcggagatctggatagcggccgcgaggtggaggggaagactttgaggacacctgtggagagctcgaattccagaacgatgaaatagt

SEQ ID NO: 18 - DNA, 569 bp, Artificial Sequence
  Full sequence of perfect circmR1 miR-20b and miR-106a
gttggagcagttggaagtcttattgtacaacatgcttcgattaagtctcccacccaaccgcggggaagatccctacctgcactatgagcactttgtcagcagctgtgctacctgcactgtaagcacttttactcgctattagctacctgcactatgagcactttgactcctatcgtactacctgcactgtaagcactttttcatgactaccgctacctgcactatgagcactttgctataagaagcactacctgcactgtaagcacttttattcggagatccctacctgcactatgagcactttgtcagcagctgtgctacctgcactgtaagcacttttactcgctattagctacctgcactatgagcactttgactcctatcgtactacctgcactgtaagcactttttcatgactaccgctacctgcactatgagcactttgctataagaagcactacctgcactgtaagcacttttattcggagatctggatagcggccgcgaggtggaggggaagactttgaggacacctgtggagagctcgaattccagaacgatgaaatagt

SEQ ID NO: 19 - DNA, 20 bp, Artificial Sequence
  5′ Intron forward primer
tagaatcgccactcctgcat

SEQ ID NO: 20 - DNA, 20 bp, Artificial Sequence
  5′ Intron reverse primer
ttgggtgggagacttaatcg

SEQ ID NO: 21 - DNA, 20 bp, Artificial Sequence
  3′ Intron forward primer
gaggtggaggggaagacttt

SEQ ID NO: 22 - DNA, 20 bp, Artificial Sequence
  3′ Intron reverse primer
tagaatcgccactcctgcat

SEQ ID NO: 23 - DNA, 22 bp, Artificial Sequence
  miR-132 perfect binding site
cgaccatggctgtagactgtta

SEQ ID NO: 24 - DNA, 21 bp, Artificial Sequence
  miR-132 bulged binding site
cgaccatggctcagactgtta

SEQ ID NO: 25 - DNA, 22 bp, Artificial Sequence
  miR-212 perfect binding site
tggccgtgactggagactgtta

SEQ ID NO: 26 - DNA, 21 bp, Artificial Sequence
  miR-212 bulged binding site
tggccgtgactccgactgtta

SEQ ID NO: 27 - DNA, 12 bp, Artificial Sequence
  Spacer 1
tcagcagctgtg

SEQ ID NO: 28 - DNA, 12 bp, Artificial Sequence
  Spacer 2
actcctatcgta

SEQ ID NO: 29 - DNA, 12 bp, Artificial Sequence
  Spacer 3
ctataagaagca

SEQ ID NO: 30 - DNA, 12 bp, Artificial Sequence
  Spacer 4
actcgctattag

SEQ ID NO: 31 - DNA, 12 bp, Artificial Sequence
  Spacer 5
tcatgactaccg

SEQ ID NO: 32 - DNA, 12 bp, Artificial Sequence
  Spacer 6
attcggagatcc

SEQ ID NO: 33 - DNA, 20 bp, Artificial Sequence
  GAPDH Human forward primer
agccacatcgctcagacacc

SEQ ID NO: 34 - DNA, 19 bp, Artificial Sequence
  GAPDH Human reverse primer
gcccaatacgaccaaatcc

SEQ ID NO: 35 - DNA, 21 bp, Artificial Sequence
  18S Mouse forward primer
ttgacggaagggcaccaccag

SEQ ID NO: 36 - DNA, 21 bp, Artificial Sequence
  18S Mouse reverse primer
gcaccaccacccacggaatcg

SEQ ID NO: 37 - DNA, 20 bp, Artificial Sequence
  Nppa Mouse forward primer
tcggagcctacgaagatcca

SEQ ID NO: 38 - DNA, 20 bp, Artificial Sequence
  Nppa Mouse reverse primer
gtggcaatgtgaccaagctg

SEQ ID NO: 39 - DNA, 22 bp, Artificial Sequence
  Nppb Mouse forward primer
gctgctggagctgataagagaa

SEQ ID NO: 40 - DNA, 25 bp, Artificial Sequence
  Nppb Mouse reverse primer
aggtcttcctacaacaacttcagtg

SEQ ID NO: 41 - DNA, 19 bp, Artificial Sequence
  Myh6 Mouse forward primer
ctacaagcgccaggctgag

SEQ ID NO: 42 - DNA, 21 bp, Artificial Sequence
  Myh6 Mouse reverse primer
tggagaggttattcctcgtcg

SEQ ID NO: 43 - DNA, 22 bp, Artificial Sequence
  Myh7 Mouse forward primer
agcattctcctgctgtttcctt

SEQ ID NO: 44 - DNA, 21 bp, Artificial Sequence
  Myh7 Mouse reverse primer
tgagccttggattctcaaacg

SEQ ID NO: 45 - RNA, 23 nt, Artificial Sequence
  hsa-miR-17-5p
caaagugcuuacagugcagguag

SEQ ID NO: 46 - RNA, 23 nt, Artificial Sequence
  hsa-miR-18a-5p
uaaggugcaucuagugcagauag

SEQ ID NO: 47 - RNA, 23 nt, Artificial Sequence
  hsa-miR-20b-5p
caaagugcucauagugcagguag

SEQ ID NO: 48 - RNA, 23 nt, Artificial Sequence
  hsa-miR-106a-5p
aaaagugcuuacagugcagguag

SEQ ID NO: 49 - RNA, 23 nt, Artificial Sequence
  hsa-miR-17-5p perfect binding site
cuaccugcacuguaagcacuuug

SEQ ID NO: 50 - RNA, 23 nt, Artificial Sequence
  hsa-miR-18a-5p perfect binding site
cuaucugcacuagaugcaccuua

SEQ ID NO: 51 - RNA, 23 nt, Artificial Sequence
  hsa-miR-20b-5p perfect binding site
cuaccugcacuaugagcacuuug

SEQ ID NO: 52 - RNA, 23 nt, Artificial Sequence
  hsa-miR-106a-5p perfect binding site
cuaccugcacuguaagcacuuuu